(19) United States
(12) Patent Application Publication
(10) Pub. No.: US 2024/0132530 A1
(43) Pub. Date: Apr. 25, 2024

(54) A HIGHLY EFFICIENT GLYCOSYLATION CHEMISTRY ENABLED BY A DIRECTING GROUP THAT IS PART OF THE ANOMERIC LEAVING GROUP

(71) Applicant: The Regents of the University of California, Oakland, CA (US)

(72) Inventors: Liming Zhang, Goleta, CA (US); Xu Ma; Zhitong Zheng, Santa Barbara, CA (US)

(73) Assignee: The Regents of the University of California, Oakland, CA (US)

(21) Appl. No.: 18/262,878

(22) PCT Filed: Jan. 28, 2022
(86) PCT No.: PCT/US22/14381

§ 371 Date: Jul. 25, 2023

Related U.S. Application Data
(60) Provisional application No. 63/142,630, filed on Jan. 28, 2021.

(51) Int. Cl. C07H 1/00

(52) U.S. Cl. CPC C07H 1/00

(57) ABSTRACT

Broadly applicable and stereoselective formation of glycosidic linkage remains challenging yet of critical importance in giycoscience. By developing an SN2 glycosylation, this work advances a general solution to this challenge via stereoinversion at the anomeric position of glycosyl ester donors. This SN2 process is enabled by a basic directing-group in the leaving-group, which is activated by a cationic gold catalyst or any other electrophilic reagent. Unlike all the reported directing group approaches, this strategy is applicable to any glycosyl donors—a long sought-after yet unmet goal in carbohydrate chemistry; moreover, the basic directing-group upon glycosylation is lost as part of the leaving-group and hence traceless in the glycoside products, therefore avoiding potential complications in downstream transformations. Highly selective construction of glycosidic bonds including challenging 1,2-cis glycosidic bonds is achieved in excellent yields. The strategy is applied iteratively to access oligosaccharides and can distinguish alcohols with different steric hindrance.

### CROSS REFERENCE TO RELATED APPLICATIONS

This application claims the benefit under 35 U.S.C. Section 119(e) of co-pending and commonly-assigned U.S. Provisional Patent Application No. 63/142,630, filed Jan. 28, 2021, by Liming Zhang, Xu Ma, and Zhitong Zheng, entitled “A HIGHLY EFFICIENT GLYCOSYLATION CHEMISTRY ENABLED BY A DIRECTING GROUP THAT IS PART OF THE ANOMERIC LEAVING GROUP,” Docket No. (30794.0796USP1), which application is incorporated by reference herein.

### STATEMENT REGARDING FEDERALLY SPONSORED RESEARCH AND DEVELOPMENT

This invention was made with Government support under Grant (or Contract) No. U01 GM125289, awarded by the National Institutes of Health (NIH). The Government has certain rights in this invention.

### BACKGROUND OF THE INVENTION

### 1. Field of the Invention

The present disclosure relates methods and compositions of matter useful in the synthesis of carbohydrates using a glycosylation process.

### 2. Description of the Related Art

(Note: This application references a number of different references as indicated throughout the specification by one or more reference numbers in parentheses, e.g. (x). A list of these different publications ordered according to these reference numbers can be found below in the section entitled “References.” Each of these publications is incorporated by reference herein.

Carbohydrates serve essential biological functions such as energy storage, structural components, and primary metabolites and play key roles in a broad range of biological processes such as signal transduction, fertilization, metathesis, cell-cell adhesion, and immune responses. The exceptional structural complexity of oligosaccharides themselves or in conjugation with other biomolecules such as natural products, lipids, and proteins poses significant challenges to their synthesis and hinders the study of their biological functions.

The main challenge arisen from the construction of each glycosidic bond in oligosaccharide synthesis¹⁻³ and glycosylated structures (e.g., glycolipids, glycoproteins, proteoglycans, glycosylated natural products, and any compounds containing a sugar moiety) is how to control the anomeric configuration formed from a diverse array of glycosyl donors and acceptors. The glycosylation reaction typically occurs via either an SN1 or SN2 pathway (FIG. 1A), with the former much more prevalent and in principle leading to the formation of both α and β anomers.⁴ Remarkable advances in stereoselective glycosylation reactions have been achieved and especially in the context of the formation of 1,2-trans glycosidic bonds via neighboring group assistance (FIG. 1B). In contrast, the formation of 1,2-cis glycosidic bond remains challenging,^{5,6} and a much-needed strategy that is applicable to every sugar type has yet to be discovered. Among various innovative methods partially addressing this challenge (FIG. 1C) are the neighboring group assistance, the remote group assistance, intramolecular aglycon delivery, steric shielding and conformation restriction, which employ glycosyl donors featuring a combination of specific O-protecting group(s) and sugar configurations that enable controlling of facial selectivity in acceptor attack via an initially formed oxocarbenium intermediate. These strategies, however, suffer from several common drawbacks/limitations: a) the need to match sugar types and specific O-protecting groups (PG) limits their generality; b) the need to install a specific PG, which is often special and distinct from common PGs, inevitably complicates donor synthesis with regard to protecting group installation/manipulation and diminish synthetic flexibility and step economy; c) such a protecting group or modification is carried into the glycoside product, which may complicate subsequent glycosylation or require additional PG manipulation. Alternative strategies toward 1,2-cis glycosides relying on solvent effects or halide ion catalysis are also limited by applicable scope. What is needed then, are improved. methods of performing glycosylation. The present disclosure satisfies this need.

### BRIEF DESCRIPTION OF THE DRAWINGS

Referring now to the drawings in which like reference numbers represent corresponding parts throughout:

FIG. 1. Strategies for stereoselective glycosylation: (A) general mechanism considerations: SN1 vs. SN2 pathways; (B) neighboring-group assistance for the construction of 1,2-trans glycosides. (C) approaches to 1,2-cis glycosides. (D) general design according to one or more embodiments described herein.

FIG. 2A-2C. Implementing the traceless directing group design

FIG. 3. Reaction scope with β-D-glucopyranosyl donors.

FIG. 4A-4B. Reaction scope with other monosaccharide donors.

FIG. 5A-5C. Applications in the synthesis of oligosaccharides.

FIG. 6A-6B. Employing a naphthoate-based leaving group for SN2 glycosylation, wherein FIG. 6A illustrates a general strategy, FIG. 6B illustrates the highly stereoselective formation of either alpha or beta glycoside via SN2 glycosylation, and FIG. 6C illustrates additional examples of SN2 glycosylation

FIG. 7 illustrates the employment of aminoalkyne as the linker in the leaving group for SN2 glycosylation.

FIG. 8A-8C illustrate example donor compounds, wherein FIG. 5A illustrates donor structure of type A, FIG. 8B illustrates donor structure of type B, and FIG. 8C illustrates donor structure of type C.

FIG. 9A. Flowchart illustrating a method of forming a carbohydrate.

FIG. 9B is a schematic illustrating a carbohydrate comprising branched chains, according to a first example.

FIG. 9C is a schematic illustrating a carbohydrate comprising branched chains, according to a second example.

FIG. 10. Example synthesizer for a solid synthesis process.

FIG. 11. Flowchart illustrating a method of making a carbohydrate according to one or more embodiments described herein.

FIG. 12. Example hardware environment.

FIG. 13. Example cloud or network environment.

### SUMMARY OF THE INVENTION

A highly stereoselective construction of 1,2-cis glycosidic bonds that is applicable to every donor sugar type has yet to be developed despite over a century of intense research in this area (i.e., carbohydrate chemistry). On the other hand, glycobiology and glycoscience, in general, depend on efficient access to oligosaccharides of defined yet diverse configurations and connectivities. This deficiency hinders the development of automation in carbohydrate synthesis, despite some recent progress. In this disclosure, we describe a solution in addressing this long-standing challenge via an SN2 strategy featuring a traceless directing group on the anomeric leaving group. The highlight of our approach is to use a directing group to promote the acceptor attack at the backend of the anomeric carbon-leaving group bond (hence realizing an SN2 process) and to attach the directing group to the leaving group. The latter feature makes the directing group traceless in the glycoside products and permits the strategy to be of general applicability as the installation of the directing group and hence the adaptation of the strategy is not limited to any specific glycosyl donor, which is otherwise the case in the current state of the art in the construction of 1,2-cis glycosides. Several implementations of this strategy are developed and disclosed here. The reaction scope studies demonstrate that indeed high levels of 1,2-cis selectivity along with excellent yields are achievable; moreover, some of the implementations can achieve highly stereoselective synthesis of 1,2-trans glycosides and other glycosidic linkages found in 2-deoxyglycosides, sialosides and unnatural glycosides via the stereoinversion of the corresponding glycosyl donors in an SN2 process. This chemistry can be applied to both pyranoside and furanoside synthesis. The process can also be implemented using solid synthesis.

Example methods, compositions, and synthesizers according to embodiments described herein include, but are not limited to, the following.
1. Several or one or more implementations of the SN2 glycosylation strategy for carbohydrate synthesis, or a method for making a carbohydrate comprising:
(a) forming one or more glycosidic bonds between a glycosyl donor—a compound donating its saccharide moiety to the oxygen atom of a hydroxyl group in an acceptor—and an acceptor—a compound possessing a hydroxyl group and using its hydroxyl oxygen to accept the sugar moiety from the donor—, comprising:
(i) obtaining a donor compound comprising:
the saccharide moiety covalently bonded to a leaving group; and
a basic group covalently bonded to the leaving group;
(ii) activating the donor leaving group by an electrophile in a presence of an HO group-bearing acceptor so as to form an activated leaving group in the donor undergoing an SN2 reaction comprising:
the basic group on the leaving group forming a hydrogen bond with the acceptor hydroxyl group; and
the formation of the hydrogen bond facilitating a nucleophilic attack by the acceptor hydroxyl group, the nucleophilic attack breaking the covalent bond between the activated leaving group and the donor saccharide moiety; and
the hydroxyl group forming a new glycosidic bond with the donor saccharide moiety in a substitution of the leaving group;
so that the carbohydrate comprising the one of the glycosidic bonds between the donor saccharide moiety and the hydroxyl group of an acceptor is made.
2. The method of example 1, wherein the carbohydrate comprises an oligosaccharide comprising a chain of a plurality of n saccharides connected by the glycosidic bonds, wherein n is an integer, the method further comprising:
repeating the forming step (a) such that:
   the carbohydrate formed in the previous step (a) comprises the acceptor compound including the hydroxyl group used to form the glycosidic bond in the next forming step (a);
   the glycosyl donor compound is delivered to the acceptor compound in each of the forming steps such that, for each of the n saccharides, the glycosidic bonds include:
   a first glycosidic bond connecting the n^{th} saccharide to the (n−1)^{th} saccharide; and
   a second glycosidic bond connecting the n^{th} saccharide to the (n+1)^{th} saccharide.
3. The method of example 3, wherein the glycosidic bonds further include a third glycosidic bond connecting the n^{th} saccharide to the (n+2)^{th} saccharide.
4. The method of examples 3 or 4, wherein the glycosidic bonds further include a fourth glycosidic bond connecting the n^{th} saccharide to the (n+3)^{th} saccharide.
5. The method of any of the examples 1-4, wherein:
the leaving group comprises an ester bonded to an alkyne, and
the basic group comprises a functional group including a basic atom that can be an oxygen, nitrogen or any other heteroatom.
6. The method of any of the examples 1-5, wherein:
the donor compound comprises either one of the structure types:

### Donor of Structure Type A

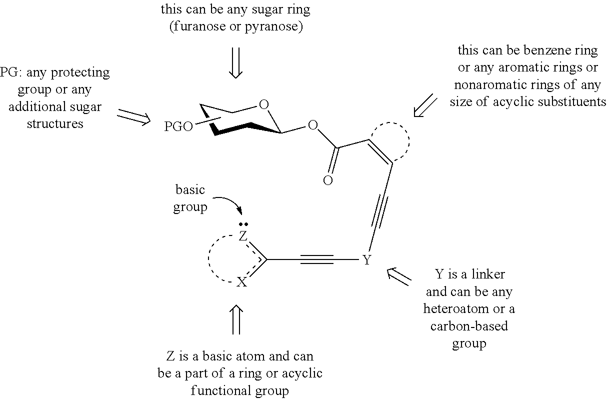

### Donor of Structure Type B

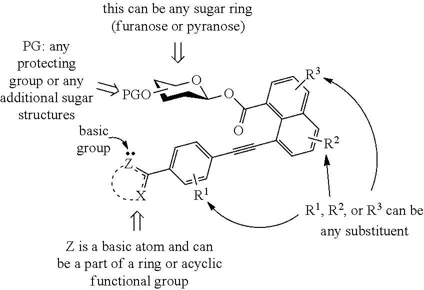

### Donor of Structure Type C

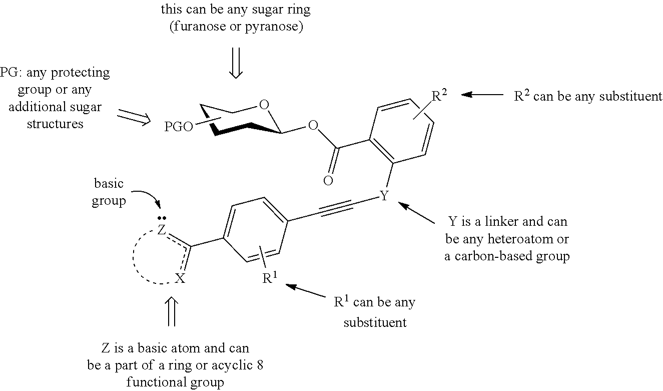

For each type, the saccharide moiety comprises the sugar ring, which can be, e.g., 5-membered, 6-membered or of any ring sizes; in one or more examples, the sugar ring can be a part of an oligosaccharide.
Z is a heteroatom, and
X is a heteroatom- or carbon-based group.
Y is a linker and can be any heteroatom or a carbon-based group
R¹, R² or R³ can be any substituents.
7. The method of any of the examples 1-6, wherein the saccharide moiety or sugar ring comprises any monosaccharide, any oligosaccharide, or any of their modified counterparts.
8. The method of any of the examples 1-7, wherein the acceptor compound comprises a protecting group covering the hydroxyl group, the method further comprising exposing the protecting group to a deprotecting agent removing the protecting group so as to expose the hydroxyl group prior to forming the glycosidic bond with the hydroxyl group.
9. The method of any of examples 2-8, wherein the oligosaccharide comprises a branched or linear chain.
10. The method of any of the examples 1-9, wherein the glycosidic bond forming comprises solid phase synthesis.
11. The method of any of the examples 1-10, wherein at least one of the acceptor compounds is connected to a solid support and one or more reagents comprising the donor compound are delivered to the solid support with at least one acceptor covalently attached.
12. The method of examples 2 and 11, wherein the acceptor compound is the first acceptor in the chain.
13. The method of any of the examples 8-10, wherein the acceptor compound is connected to the solid support via a linker structure.
14. The method of any of the examples 10-13, wherein the solid support comprises a polymer or polymer resin.
15. The method of any of the examples 1-14, wherein the glycosidic bond forming further comprises providing a catalyst or activation reagent (e.g., an electrophilic halogen) activating the leaving group toward the nucleophilic attack by the acceptor hydroxyl group.
16. The method of any of the examples 2-15, wherein the oligosaccharide comprises a 1,2-cis glycosidic or any other glycosidic bond.
17. A carbohydrate synthesized according to method of any of the examples 1-16.
18. The carbohydrate of example 17, wherein the carbohydrate does not comprise a protecting group covering the hydroxyl group, the protecting group removed by exposure to a deprotecting agent removing the protecting group.
19. A synthesizer for making a carbohydrate or a glyco-conjugated compound, comprising:
a solid support for an acceptor comprising a hydroxyl group (“acceptor hydroxyl group”);
a computer executing an algorithm controlling a reaction forming one or more glycosidic bonds between a donor and the acceptor; and
a delivery system for delivering reagents to the solid support in accordance with the algorithm, the reagents including:
a donor compound comprising:
   the saccharide covalently bonded to a leaving group; and
   a basic group covalently bonded to the leaving group;
an electrophilic catalyst or activation reagent activating an electrophilicity of the leaving group so as to form an activated leaving group in a presence of the acceptor hydroxyl group, the electrophilicity allowing the reaction comprising an SN2 reaction comprising:
   the basic group on the leaving group forming a hydrogen bond with the acceptor hydroxyl group; and
   the acceptor hydroxyl group performing a nucleophilic attack breaking the covalent bond between the activated leaving group and the saccharide moiety of the donor; and
   the acceptor hydroxyl group forming one of the glycosidic bonds with the saccharide moiety of the donor in a SN2 substitution of the activated leaving group;
so that the carbohydrate comprising the one of the glycosidic bonds between the saccharide moiety of the donor and the acceptor hydroxyl group is made.
20. The synthesizer of example 19 configured to perform the method of any of the examples 1-18.
21. The synthesizer of any of the examples 19-20, wherein the acceptor compound comprises a protecting group covering the hydroxyl group and the delivery system further delivers a deprotecting agent removing the protecting group so as to expose the hydroxyl group and form the carbohydrate without the protecting group.
22. A composition of matter useful as a glycosyl donor in glycosylation reaction, comprising:
a compound comprising:
a saccharide covalently bonded to a leaving group, the leaving group comprising a moiety or functionality that can be activated to form an activated leaving group exhibiting electrophilicity; and
a basic group covalently bonded to the leaving group, the basic group forming a hydrogen bond with an acceptor hydroxyl group so that the acceptor hydroxyl group performs a nucleophilic attack breaking the covalent bond between the activated leaving group and the saccharide moiety of the donor in a presence of the electrophilicity.
23. The composition of matter of example 22, wherein the moiety or functionality in the leaving group comprises an ester covalently bonded to an alkyne.
24. The composition of matter of any of the examples 22-23, wherein the basic group includes a hydrocarbon derivative comprising at least one of oxygen or nitrogen or any other heteroatom.
25. The composition of matter of examples 22-24, wherein the basic group is connected to the alkyne directly or indirectly.
26. The composition of matter of any of the examples 22-25, wherein:
the compound has these structures:

### Donor of Structure Type A

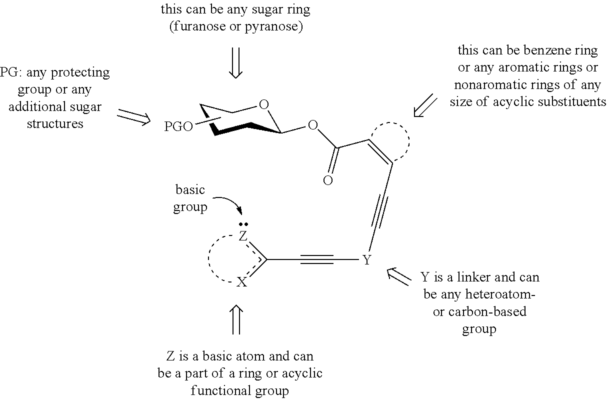

### Donor of Structure Type B

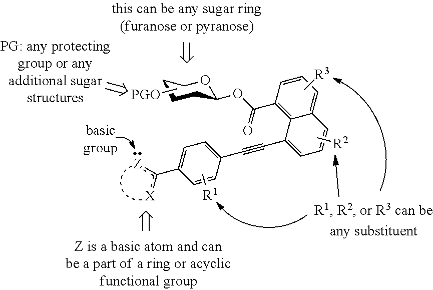

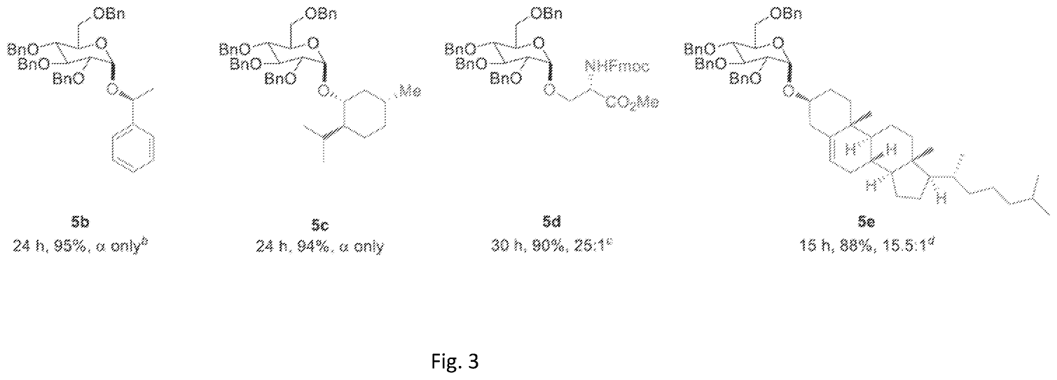

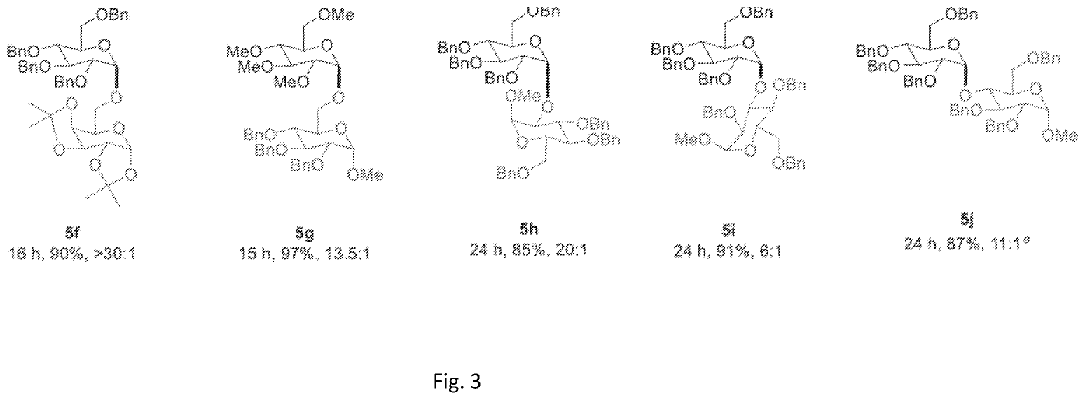

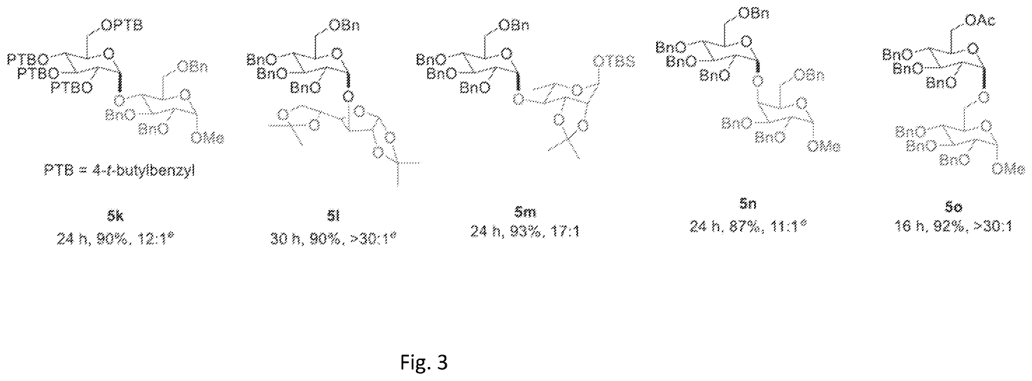

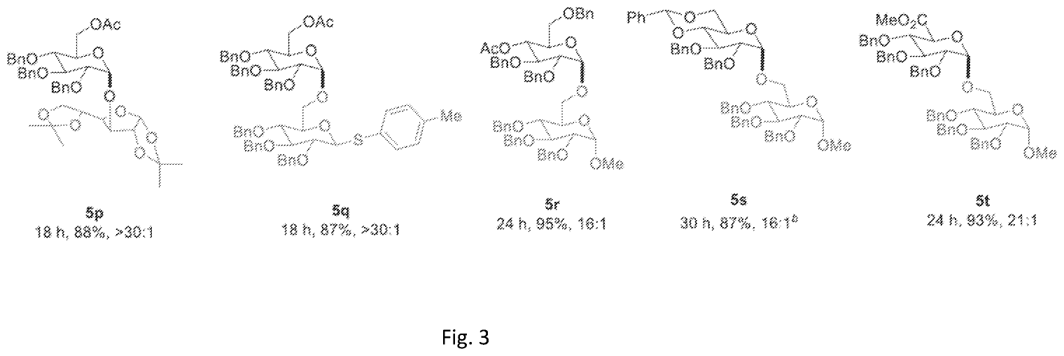

### Donor of Structure Type C

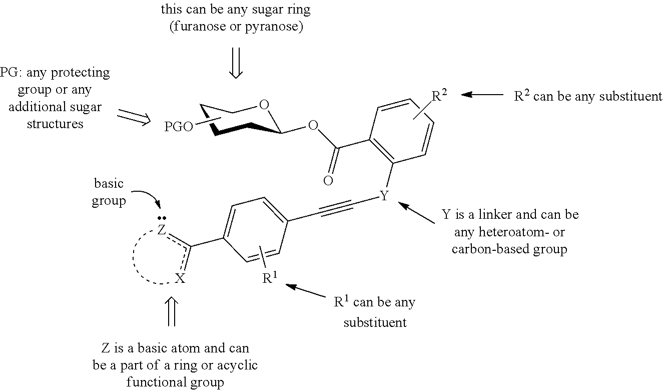 [0094] the saccharide comprises the sugar ring,
Z is a heteroatom, and
X is a heteroatom- or carbon-based group.
Y is a linker and can be any heteroatom or a carbon-based group
R¹, R² or R³ can be any substituents.
27. The composition of matter of any of the examples 22-26, wherein the compound comprises the donor compound in any of the examples 1-18.
28. The composition, synthesizer, or method of any of the examples, wherein the donor compound comprises any of the structures in FIGS. 3-6.
29. The composition of matter of any of the examples 25-27, wherein the PG group and donor can comprise a disaccharide or oligosaccharide or any sugar moiety.
30. The composition, synthesizer, or method of any of the examples 1-29, wherein the basic group comprises a phenyl, or steric bulky group.
31. The composition, synthesizer, or method of any of the examples 1-30, wherein the leaving group comprises a benzoate or aromatic carboxylate group.
32. The composition, synthesizer, or method of any of the examples 1-31, wherein the forming of the glycosidic bonds comprises combining the donor group and the acceptor group in a solvent comprising an aprotic solvent.
33. The composition, synthesizer, or method of example 32, wherein the solvent further comprises a minor, less polar, component
34. The composition, synthesizer, or method of any of the examples 1-33, wherein the forming of the glycosidic bonds comprises combining the donor compound comprising the type A donor or the type B donor and the acceptor compound in a solvent comprising trifluorotoluene.
35. The composition, synthesizer, or method of any of the examples 1-34, wherein:
the forming of the glycosidic bonds comprises providing a catalyst in a presence of an acid, the catalyst activating the leaving group toward the nucleophilic attack by the acceptor hydroxyl group;
the catalyst comprises a metal and the acid reactivates the metal so as to increase a yield of the carbohydrate.
35. The composition, synthesizer, or method of example 35, wherein the acid comprises bis(trifluoromethanesulfonyl)imide.
36. The composition, synthesizer, or method utilizing the donor compound of type A in example 26, wherein Y comprises a cyclopropyl.
37. The composition, synthesizer, or method of any of the examples 30-36, wherein the composition of the basic group and the leaving group increases selectivity for an alpha or beta type of the carbohydrate.
38. The composition, synthesizer, or method of any of the examples 30-37, wherein a composition of the solvent and/or the acid increases a reaction rate of the formation of the glycosidic bonds.
39. A carbohydrate, comprising:
one or more glycosidic bonds, each of the glycosidic bonds between a first saccharide moiety and a hydroxyl group connected to a second saccharide moiety, the carbohydrate manufactured by a process comprising:
(a) forming the one or more glycosidic bonds between a glycosyl donor compound and a an acceptor compound comprising the hydroxyl group, comprising:
(i) obtaining the glycosyl donor compound comprising:
the first saccharide moiety covalently bonded to a leaving group; and
a basic group covalently bonded to the leaving group;
(ii) activating the leaving group by an electrophile in a presence of the acceptor compound so as to form an activated leaving group in the glycosyl donor compound undergoing an SN2 reaction comprising:
the basic group forming a hydrogen bond with the hydroxyl group; and
formation of the hydrogen bond facilitating a nucleophilic attack by the hydroxyl group, the nucleophilic attack breaking the covalent bond between the activated leaving group and the saccharide moiety; and
the hydroxyl group forming the one of the glycosidic bonds with the first saccharide moiety in a substitution of the leaving group;
so that the carbohydrate is made.
40. The composition, synthesizer, or method of any of the examples 1-38, or any example described herein, wherein the acceptor compound is any HO group-containing compound.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

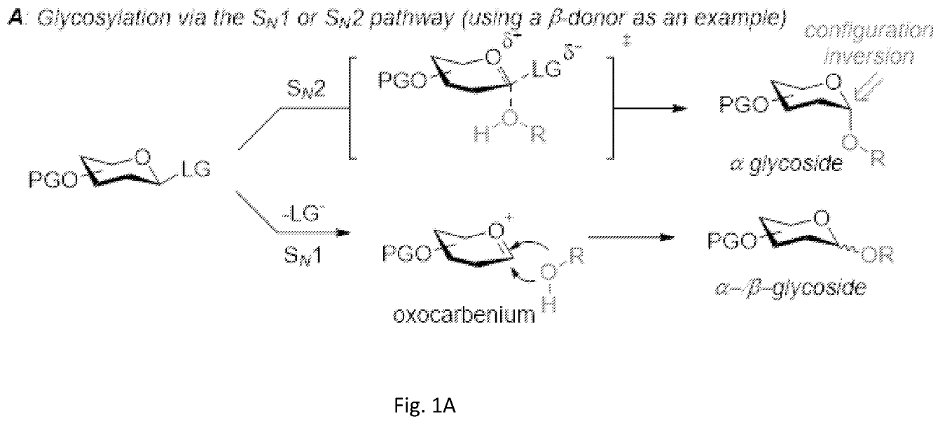

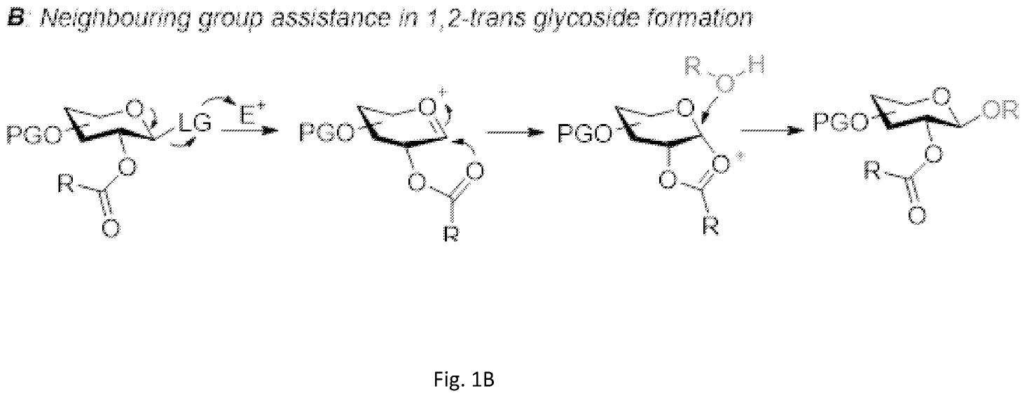

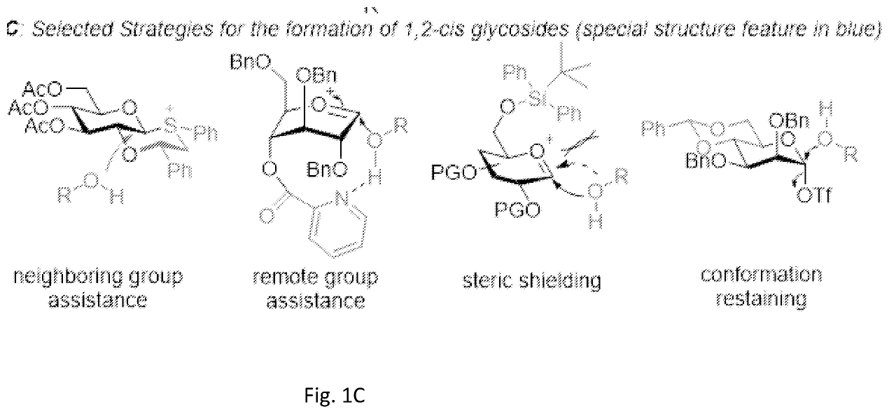

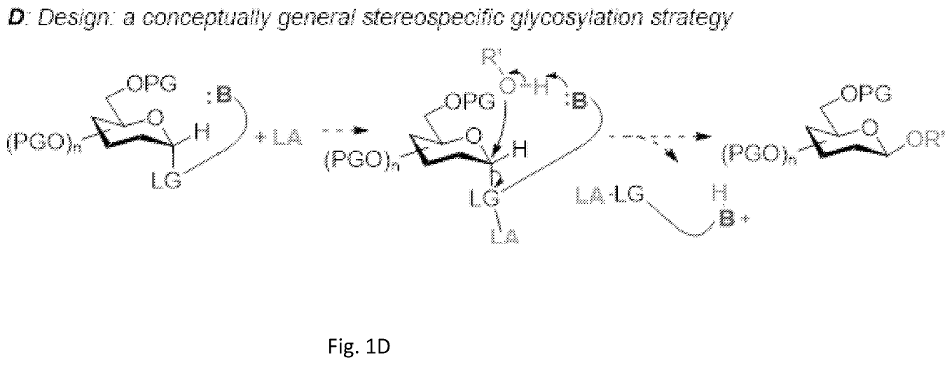

The present disclosure describes a glycosylation strategy that employs a basic group-assisted delivery of acceptor (FIG. 1D). A key feature of this design is that it permits stereoselective formation of any glycosidic bond, regardless of the donor saccharide configurations and protecting group patterns, and is distinct from all reported strategies is that the basic directing group is to be installed on the glycosyl leaving group and hence traceless in the glycoside product upon reaction. This design necessitates SN2-type glycosylation in order to harness the directing effect. By using 1,2-trans glycosyl donor, this strategy would lead to a general solution to the challenging construction of 1,2-cis glycosides.

### First Example: Glycosidic Bond Formation Using a Donor Based on the Structure Type A

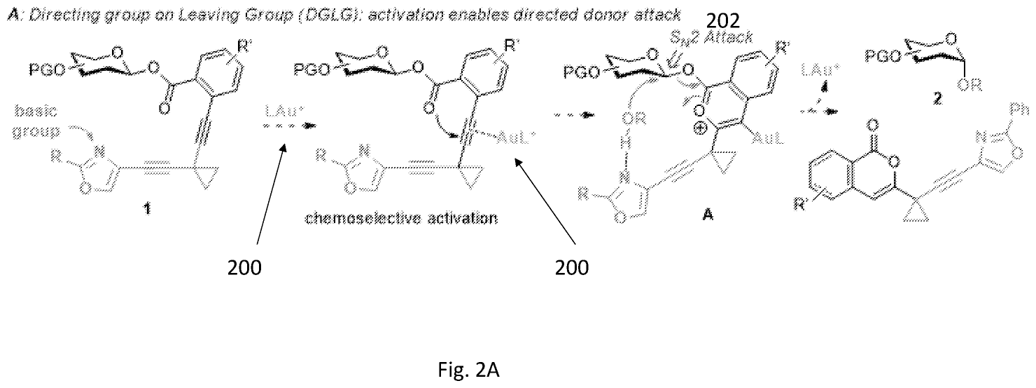

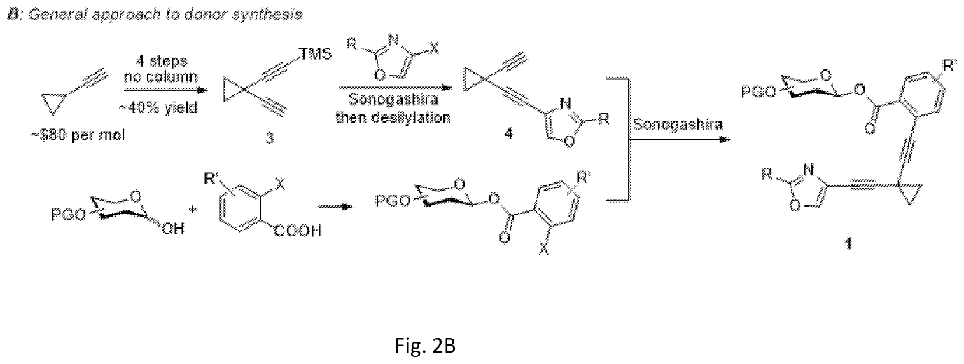

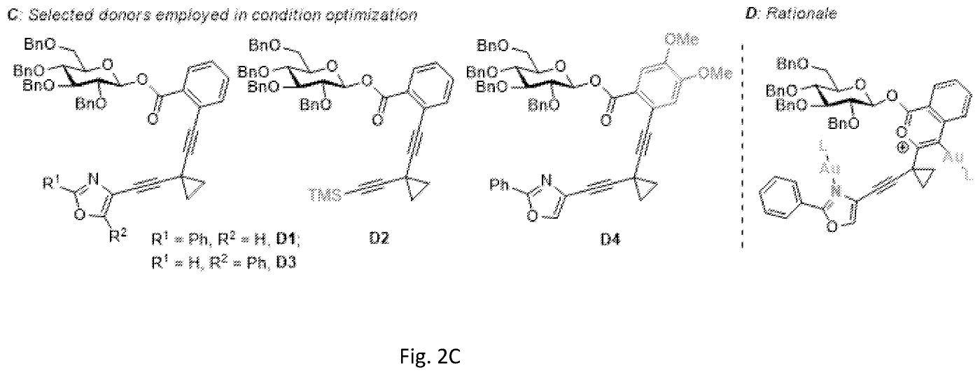

In the first implementation of the SN2 strategy, the LG is engineered based on the ortho-alkynylbenzoates used in Yu's glycosylation chemistry.⁷ As shown in FIG. 2A, in the designed donor 1, the ortho C—C triple bond is terminated by an alkynylcyclopropyl group, which features a mild basic oxazole ring. It is anticipated that its gold-promoted cyclization would deliver the isochromenylium intermediate A,⁸ which has its sidearm oxazole ring positioned to direct an alcohol acceptor at the backend of the activated anomeric C—O bond. Such delivery of the acceptor would afford the glycoside 2 with the inverted configuration at the anomeric carbon and hence realize the desired SN2 glycosylation. The gold catalyst could be regenerated upon protodeauration.

TABLE 1
Reaction discovery and optimization

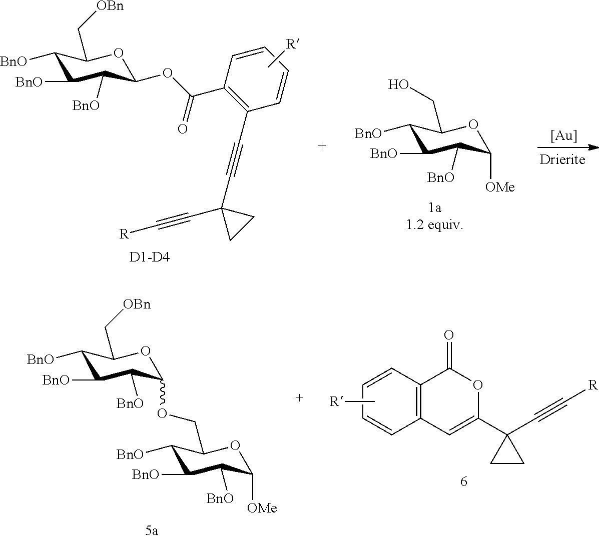

| | | catalyst | | Solvent | | | |
|---|---|---|---|---|---|---|---|
| entry | Donor | (equiv.) | Halide Abstractor (equiv.) | (conc.) | temp. /time | Conversion | Yield (α/β)^{b} |
| 1 | D1 | PPh₃AuNTf₂ (0.2) | N/A | DCM (0.02 M) | −35° C./24 h | 100% | 95% (9:1) |
| 2 | D2 | PPh₃AuNTf₂ (0.2) | N/A | DCM (0.02 M) | −35° C./24 h | 100% | 95% (1.5:1) |
| 3 | D1 | PPh₃AuNTf₂ (0.5) | N/A | DCM (0.02 M) | −35° C./12 h | 100% | 95% (2.8:1) |
| 4 | D1 | PPh₃AuNTf₂ (1.0) | N/A | DCM (0.02 M) | −35° C./12 h | 100% | 96% (1.5:1) |
| 5 | D3 | PPh₃AuNTf₂ (0.2) | N/A | DCM (0.02 M) | −35° C./24 h | <5% | <5% (N/A) |
| 6 | D4 | PPh₃AuNTf₂ (0.2) | N/A | DCM (0.02 M) | −35° C./5 h | 100% | 96% (9:1) |
| 7 | D4 | IMesAuCl (0.06) | [Ag(MeCN)₂]⁺BARF (0.05) | DCM (0.02 M) | 0° C./16 h | 62% | 62% (>20:1) |
| 8 | D4 | IMesAuCl (0.06) | [Ag(MeCN)₂]⁺BARF (0.05) | PhCF₃ (0.02 M) | 0° C./16 h | 100% | >99% (>20:1) |
| 9 | D4 | IMesAuCl (0.06) | [Ag(MeCN)₂]⁺BARF (0.05) | PhCF₃ (0.02 M) | −15° C./24 h | 41% | 41% (>30:1) |
| 10 | D4 | IMesAuCl (0.06) | AgSbF₆ (0.05) | PhCF₃ (0.02 M) | 0° C./24 h | 80% | 80% (10:1) |
| 11 | D4 | IMesAuCl (0.06) | AgNTf₂ (0.05) | PhCF₃ (0.02 M) | 0° C./24 h | 92% | 92% (14:1) |
| 12 | D4 | IMesAuCl (0.06) | [Ag(MeCN)₂]⁺BARF (0.05) | 0.08M^{c} | −15° C./15 h | 100% | >99%^{d} (27:1) |
| 13 | D4^{e} | IMesAuCl (0.06) | [Ag(MeCN)₂]⁺BARF (0.05) | 0.08M^{c} | −15° C./15 h | 100% | >99% (>20:1) |

^{a}Reaction was stirred in a cooling bath before being quenched by ⁿBu₄NCl.
^{b}Combined yield and anomeric ratio determined by NMR with internal reference.
^{c}PhCFs and cyclohexane (v/v = 4:1) were used as the solvent.
^{d}97% yield of isolated product.
^{e}0.4 equiv. MeCN added.

The designed donors can be prepared straightforwardly. As shown in FIG. 2B, the synthesis of the known 1,1-diynylcyclopropane 3 is realized in 4 steps without column purification and can be readily scaled up.⁹ Two consecutive Sonogashira cross-couplings with the oxazole electrophile and the anomerically pure glycosyl ortho-halobenzoate would afford the target donor. Table 1 outlines the reaction discovery and conditions optimization. Initial experiments began with the construction of methyl D-glucopyranosyl-(1→4)-α-D-glucopyranoside 5a from the β-D-glucopyranosyl ester donor D1 (FIG. 2C), which contains a 2-phenyl group on the oxazole ring, and methyl α-D-glycopyranoside 1a as the acceptor. 5a was obtained with a respectable α/β ratio of 9:1 in the presence of 20 mol % of PPh₃AuNTf₂ in methylene chloride at −35° C. (entry 1). In comparison, the donor D2 devoid of the oxazole ring led to a α/β ratio of 1.5:1 (entry 2), revealing the critical role of the basic heterocycle in enabling anomeric configuration inversion. This conclusion is further supported by the outcomes when the amount of the gold catalyst was varied. For example, the α/β ratio was lowered to 2.8:1 in the case of 50% PPh₃AuNTf₂ (entry 3) and further to 1.5:1 in the case of one equivalent of the gold catalyst (entry 4). It was reasoned that the cationic gold(I) binds to the oxazole nitrogen and thereby removes it from directing/facilitating the acceptor's backend attack (FIG. 2D). As a result, the beneficial role of oxazole appeared to be eliminated (entries 2 vs. 4). This detrimental binding of oxazole to Au(I) is even more pronounced with the donor D3, of which the oxazole is devoid of substitution at its 2 position and hence presents an unhindered ring nitrogen for coordination. In this case, the gold catalyst appeared to be largely sequestered and little reaction was detected (entry 5). Upon further donor and conditions optimization, it was discovered that the donor D4 bearing two electrons donating methoxy groups on its benzoate moiety permits a faster reaction and at the same time maintaining the same level of α-selectivity (entry 6). With IMesAu⁺ generated from IMesAuCl and [Ag(MeCN)₂]⁺ BARF⁻ as the catalyst in 5 mol %, the reaction was sluggish even at 0° C. due to the decreased acidity and the lower loading of the catalyst. With 62% conversion after 16 h, the α/β selectivity was however improved to >20:1 (entry 7). Changing the reaction solvent from methylene chloride to trifluorotoluene resulted in complete consumption of the substrate and near quantitative yield while maintaining excellent α-selectivity (entry 8). Lowering the reaction temperature led to an even better selectivity but at the cost of reaction rate (entry 9). Substituting BARF⁻ by SbF₆⁻ (entry 10) or NTf₂⁻ (entry 11) also led to incomplete reaction. The solvent effect, as evident from entries 7 and 8, was further examined. It was discovered that the isochromen-1-one byproduct 6 is polar and crystalline and has a significantly higher solubility in DCM (>0.02 M at −15° C.) than in PhCF₃ (˜0.0056 M at −15° C.). It is reasoned that the slower reaction rate in DCM is partly due to the coordination of Au(I) by the oxazole nitrogen of 6. On the contrary, in the less polar PhCF₃, most of 6 precipitates out from the reaction and hence does not slow the reaction much. To this end, with a mixture of PhCF₃ and even less polar cyclohexane (v/v=4:1) and at an increased concentration (0.08 M), the reaction was substantially accelerated and proceeded to completion after 15 h even at −15° C. (entry 12). The reaction was again quantitative and highly α-selective. Under these optimal conditions, most of the byproduct crashed out during the reaction while a significant amount of the donor and almost all the acceptors were dissolved. The addition of 0.4 equiv. of MeCN to the reaction showed no adverse effect on selectivity and yield, confirming that acetonitrile in [Ag(MeCN)₂]⁺ BARF⁻ is inconsequential (entry 13).

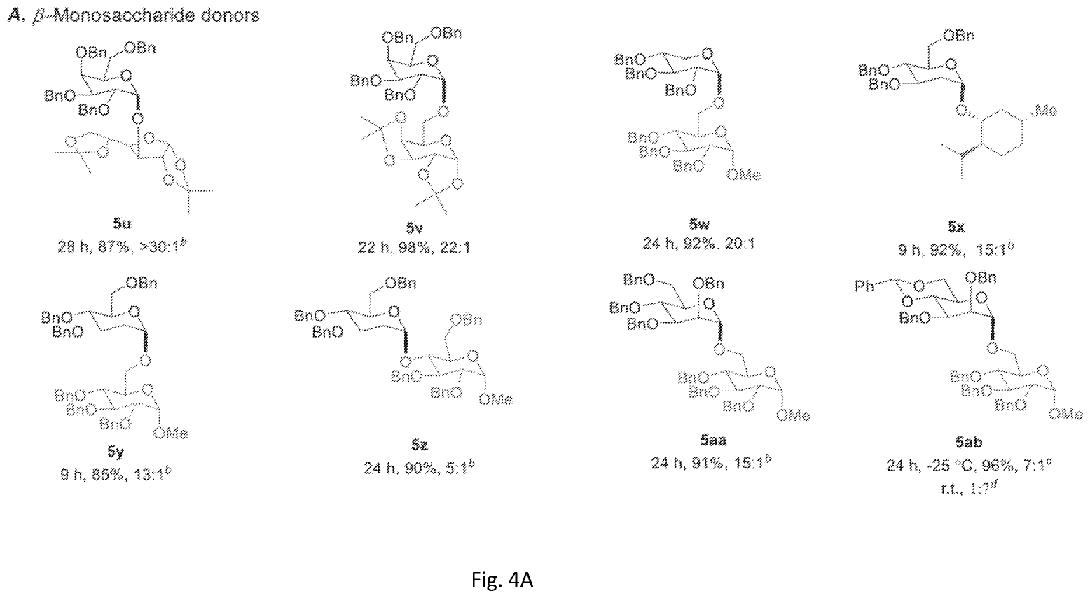

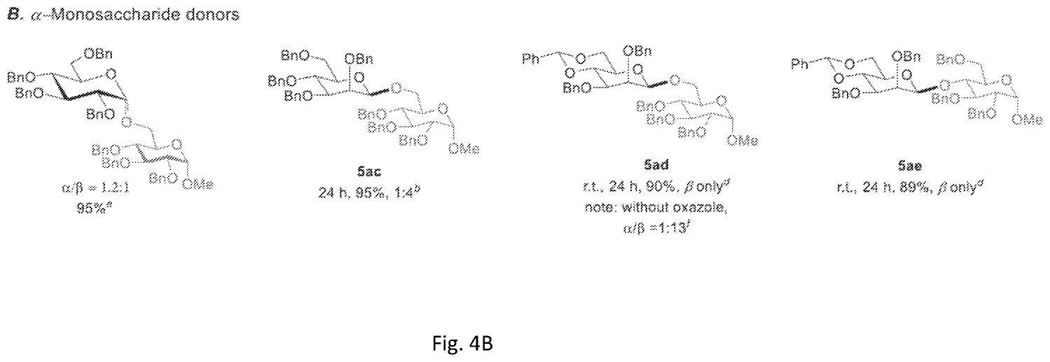

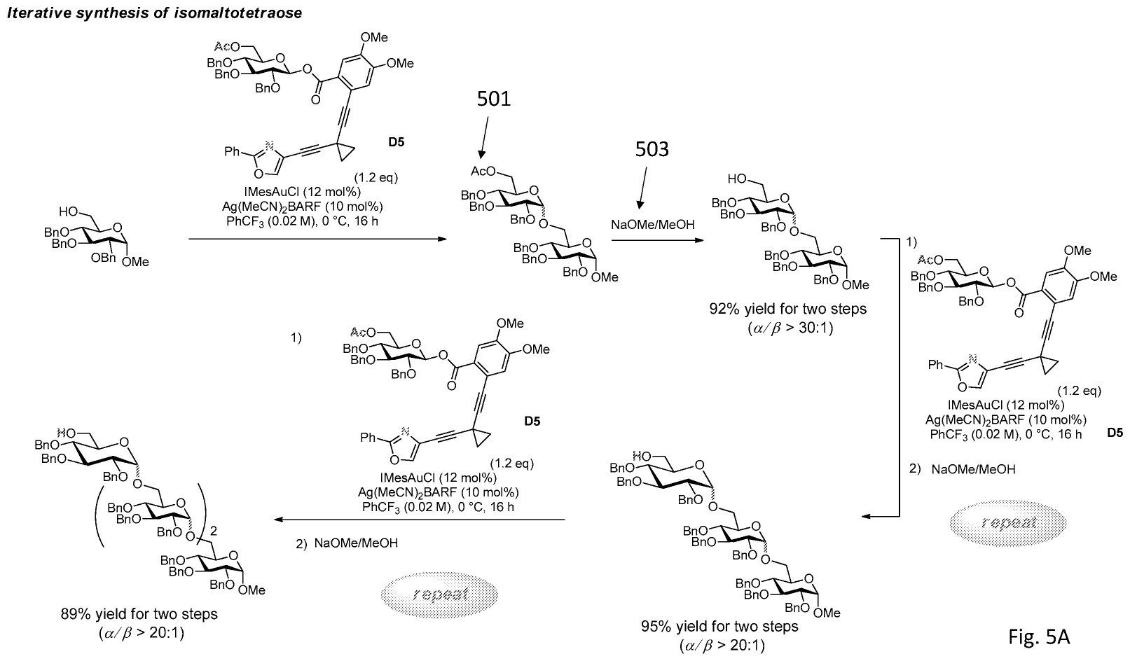

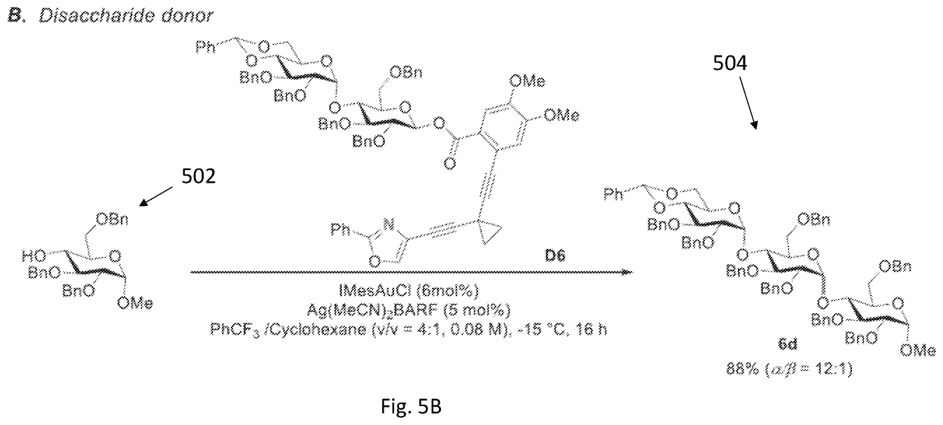

The optimized reaction conditions (i.e., Table 1, entry 12) are applied to a range of acceptors by using D4 as the donor. As shown in FIG. 3, chiral alcohols like (R)-1-phenylethanol and L-menthol proceed with excellent yields and nearly exclusive α selectivity (FIGS. 3, 5b and 5c). L-serine esters and cholesterol were running at different solvent due to their poor solubility in the mixed solvent system. The reaction with L-serine ester was slow and hence run at 0° C. for 30 hours. Nevertheless, the yields and α-selectivities remain high (5d and 5e). The reaction of the galactopyranose-based primary alcohol acceptor proceeded in 90% yield and with >30:1 α/β ratios (5f). With methyl groups replacing the benzyl groups in D4, the selectivity was diminished to 13.5:1 (5g). This was attributed to the fact that methyl is less inductively electron-withdrawing than benzyl and consequently the methylated donor has a higher tendency of participating in the SN1 pathway. Tri-O-benzylated D-pyranoglucose acceptors with an unprotected secondary hydroxy group at 2-, 3-, or 4-position, respectively (5h-5j) were then examined. While the yields are all high, the α-selectivity ranges from >20:1 for the 1→2 linkage, 11:1 for the 1→4 linkage to 6:1 for the 1→3)-3 linkage. The 4-t-butylbenzyl counterpart of the donor D4, which shall be more soluble in the mixed nonpolar solvent system, led to only marginal improvements on yield and selectivity (5k). The D-glucofuranose and L-rhamnopyranose-derived acceptors also reacted well, exhibiting excellent yields and selectivities (5l and 5m), while the reaction with methyl α-O-2,3,6-tribenzylgalactopyranoside displayed a respectable 11:1 preference for the α-glycosidic linkage (5n). With readily removable acetyl replacing the O-6-benzyl group in D4, generally higher selectivity was observed (5o-5q). It is noteworthy that this gold catalysis accommodates a thioglycoside as the acceptor (5q) and thus permit downstream glycosylation via thioglycoside activation. In addition, replacing the O-4-benzyl group of D4 with acetyl posed no problem, and 5r was formed in 95% yield and an α/β ratios of 16:1, which is only marginally lower than that with D4. Further modifying D4 by installing O-4,6-benzylidene protection or oxidizing it into methyl gluconate was inconsequential, as the products from reacting with 1a, i.e., 5s and 5t, were formed in excellent yields and with high α-selectivities.

The applicability of this design to β-donors derived from other monosaccharides was then investigated. As shown in FIG. 4A, the reaction of the corresponding β-D-galactopyranosyl donor with a primary or secondary alcohol donor also exhibited excellent α selectivities, affording the 1,2-cis-galactoside 5u and 5v in excellent yields. The reaction of the xylopyranosyl donor also proceeded with excellent selectivity and in 92% yields (5w). With the 2-deoxy counterpart of D4 as the donor, the reactions of (−)-menthol and 1a exhibits ≥13:1 preference for the α-products 5x and 5y. Its reaction with the more challenging methyl 2,3,6-tri-O-benzylglucopyranoside afforded 5z in 90% yield and with a serviceable α/β ratio of 5:1. It is of note that these α-selectivities are lower than their glucose counterparts (see 5a, 5c, and 5j), which can be attributed to the more labile nature of the anomeric C—O bond. With the perbenzylated mannose donor, the α-selectivity remains good but less than that of D4, despite the product 5aa is a 1,2-trans-mannoside. With the 4,6-benzylidene-protected mannosyl donor, the glycosylation, however, exhibits lesser SN2 characteristics than in the case of 5s. Even at −25° C. , the α/β selectivity was only 7:1, despite in 96% yield. Moreover, when the reaction was run at the ambient temperature, the selectivity is reverted to the opposite, favoring the β-anomer of the product 5ab. These result reveals donor/intermediate anomeric epimerization and suggests that in comparison to the dibenzyl protection the 4,6-benzylidene results in the more labile β-leaving group in the mannosyl version of A.

This SN2 glycosylation was also explored with α-donors. As shown in FIG. 4B, there is little anomeric selectivity with the α-anomeric epimer of D4 as the donor. This reaction likely proceeds via a SN1 process, suggesting surprisingly much more labile nature of the activated leaving group at the glucosyl α-position than at the β-position. With the mannosyl perbenzylated donor, the same trend, i.e., a higher level configuration inversion is observed with the β-donor in the case of 5aa than the α-donor in the case of 5ac, is observed. However, as discussed above, with the 4,6-benzylidene-protected mannosyl α-donor, the reaction exhibited exclusive anomeric configuration inversion with both the O-6 and the more challenging O-4 glucoside acceptors (5ad and 5ae). This result is consistent with the previous observation that with the 4,6-benzylidene protection the α-leaving group in the mannosyl version of A is less labile than its β-counterpart. Notably, for the formation of 5ad, the α/β ratio was only 1:13 when the donor with its oxazole group replaced by a non-directing cyclopropyl group was employed, suggesting the operation of the directing effect besides the inherent selectivity expected based on Crich's work.¹⁰

To demonstrate the utility of this traceless directing group strategy, it was applied to the synthesis of oligosaccharides. As shown in FIG. 5A, using methyl 2,3,4-O-tribenzyl-D-glucopyranoside as the initial acceptor and the 6-acetate analog of D5 as the donor, an iterative process consisting of the glycosylation chemistry and basic hydrolysis delivered the protected isomaltotetraose 6c in 78% overall yield and excellent α-selectivities after three iterations. Alternatively, the related maltotriose derivative 6d can be accessed in one-step from the α-maltose-derived donor D6 and methyl 2,3,6-tri-O-benzyl-α-D-glucopyranoside in an excellent yield and with good α-selectivity (FIG. 5B). During these studies, it was observed there are reactivity differences between primary and secondary alcohol acceptors, with the former reacting significantly faster due to lesser steric hindrance. This phenomenon is confirmed by the reaction of methyl 2,3-di-O-benzyl-α-D-glucopyranoside with D4, in which the sterically more accessible 6-OH group is selectively glycosylated to afford the disaccharide 5af with excellent α-selectivity and in 92% yield. The further reaction with the mannose-derived donor D7 afforded the branched trisaccharide 6e in 89% yield and with an β/α ratio of >20:1. This two-step sequence was also run in one-pot without the isolation of 5af, and the overall yield is 82%.

These results reveal that this directed SN2 glycosylation works particularly well with a range of β-donors, resulting in selective formation of α-glycosides including challenging 1,2-cis-glucosides, 1,2-cis-galactosides, and 1,2-cis-xylopyranosides. For α-donors, this glycosylation exhibits moderate or poor SN2 characteristics. However, in the case of 4,6-benzylidene-protected mannose cases, the α-donor reacts with exclusive anomeric configuration inversion, affording the 1,2-cis-mannosides with excellent β-selectivity. Consequently, in complementary to the well-practiced 1,2-trans glycosylic bond formation via the anchimeric assistance, this chemistry offers a general solution to the construction of 1,2-cis glycosidic bonds while posing little restriction on donor structures.

### Second Example: Glycosidic Bond Formation Using a Donor Based on the Structure Type B

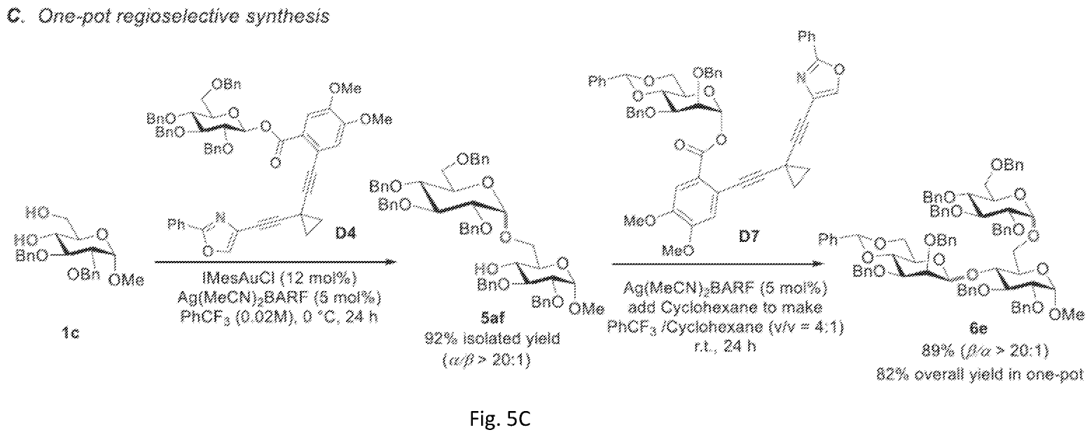

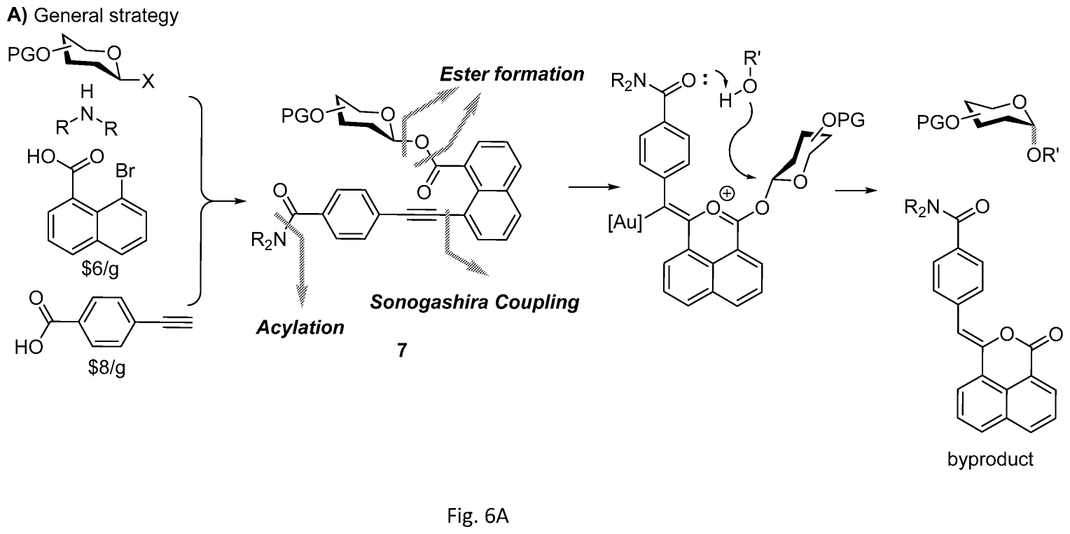

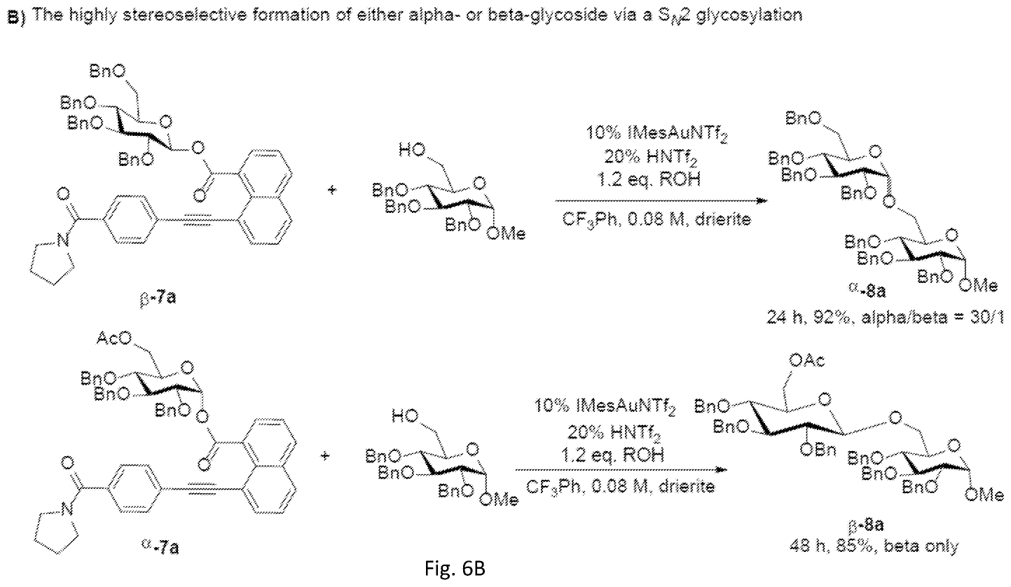

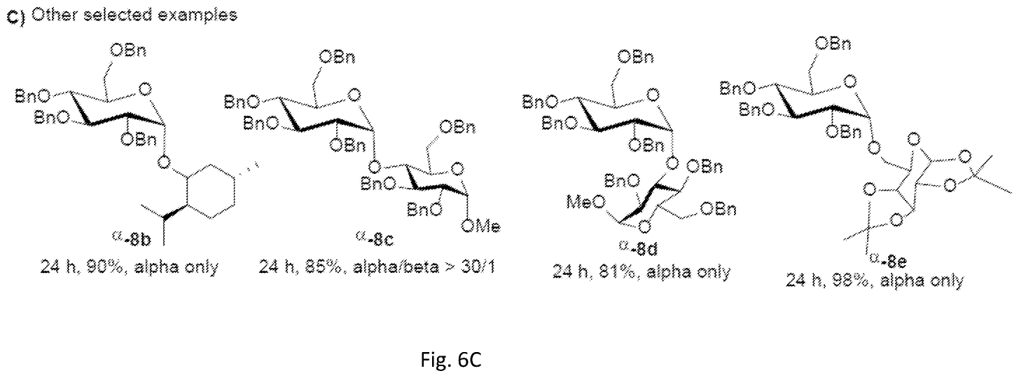

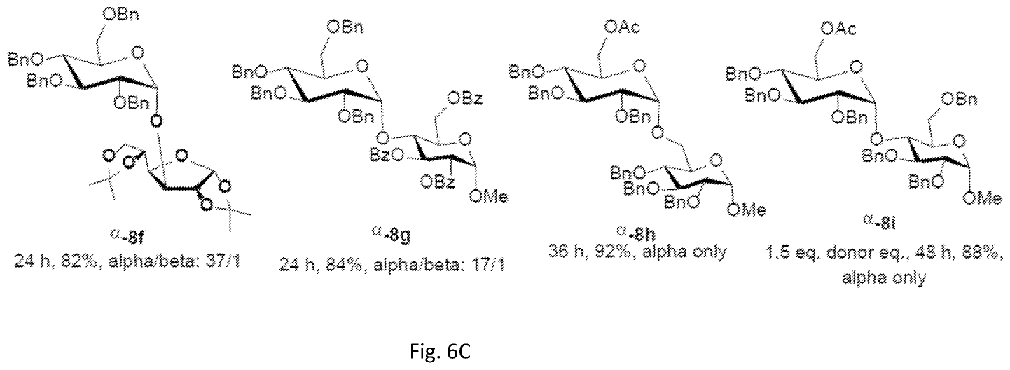

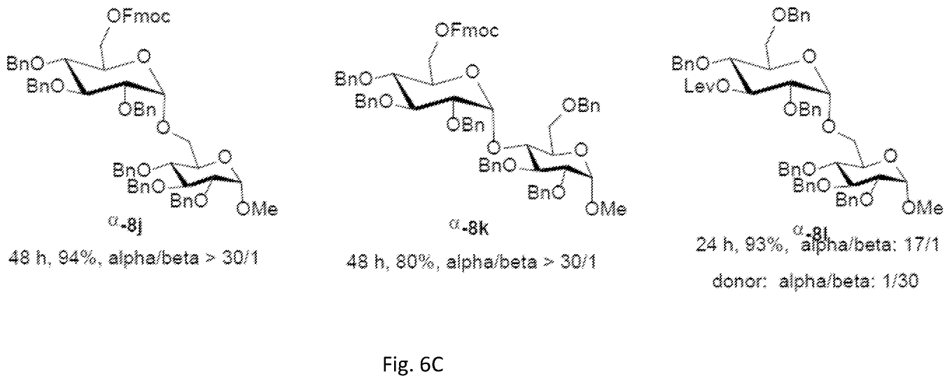

In this implementation, as shown in FIG. 6A, a naphthoate serves as the leaving group. At the C8 position of the naphthylene ring is attached an arylacetylene. The aryl group is functionalized by a para carbamide, of which the amide nitrogen is fully substituted. The glycosyl donor can be prepared in a three-step sequence from commercial materials, which entails amide formation, ester formation and at the end a Sonogashira coupling. The glycosylation reaction is catalyzed by a cationic gold complex and, analogous to First Example, in the gold-containing cyclized intermediate, the acceptor hydroxyl group forms a H-bonding interaction with the amide carbonyl oxygen. Such an interaction directs the acceptor hydroxyl group to attack the activated leaving group at the saccharide anomeric carbon. The configuration at the anomeric position is inverted in this SN2 process.

This approach exhibits high levels of stereochemical inversion at the anomeric position in the glycosidic bond forming process. As shown in FIG. 6B, the β-donor β-7a reacted with methyl 3,4,5-tri-O-benzyl-α-D-glucopyranoside to deliver the disaccharide α-8a in 92% yield and with a α/β selectivity of 30/1. Conversely, α-7a was converted to β-8a in 85% yield and with exclusive β-selelectivity.

Other examples of this approach are shown in FIG. 6C. The excellent α-selectivities achieved in the cases of α-8c, α-8d, α-8f, α-8g, α-8i, and α-8k are noteworthy and better than those observed in the cases of α-5i and α-5j in First Example. Overall, in comparison to First Example, this Example exhibits in general higher levels of stereochemical inversion at the anomeric carbon in the glycosylation process.

### Third Example: Glycosidic Bond Formation Using a Donor Based on the Structure Type C

In this implementation, the glycosyl benzoate 9 of the Structure Type C serves as the glycosyl donor. The donor benzoate moiety links to the saccharide anomeric carbon via its carboxy oxygen, and the ortho position of the benzoate benzene ring is substituted by an amino group, which is in turn substituted by an arylethynyl group. The position on the arylethynyl arene ring para to the C—C triple bond is substituted by a tertiary carboxamide. The donors of this type can be prepared via a combination of acylation, glycosyl ester formation and nucleophilic substitution reactions. Due to the electron-rich nature of the alkynylamine moiety, the electrophiles capable of activating the donor C—C triple bond toward nucleophilic attack by the benzoate carbonyl oxygen can be cationic gold, cationic silver, electrophilic halogen, or any other electrophiles. In the cyclized intermediate, the acceptor hydroxyl group forms a H-bonding interaction with the amide carbonyl oxygen. Such an interaction directs the acceptor hydroxyl group to attack the activated leaving group at the saccharide anomeric carbon, yielding a glycosidic bond. The configuration at the anomeric position is inverted in this SN2 process.

### Fourth Example: Example Donor Compounds

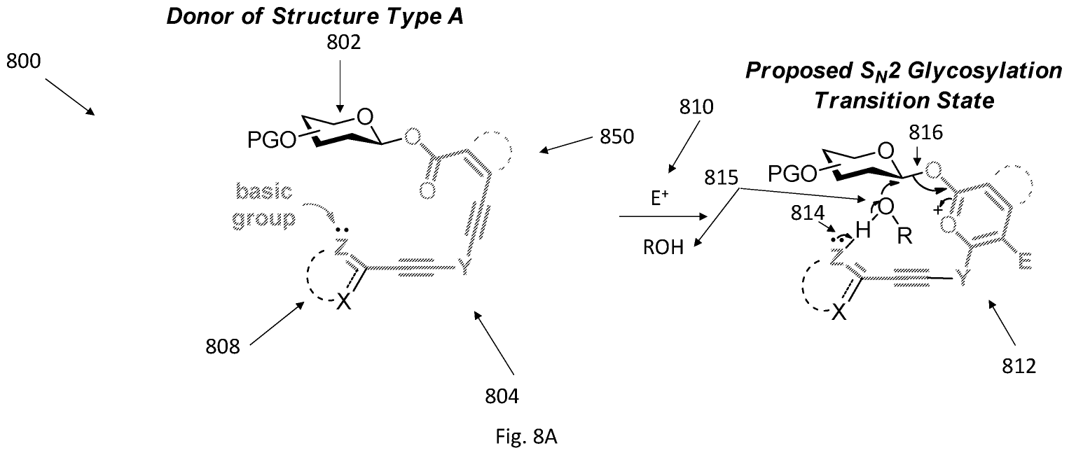

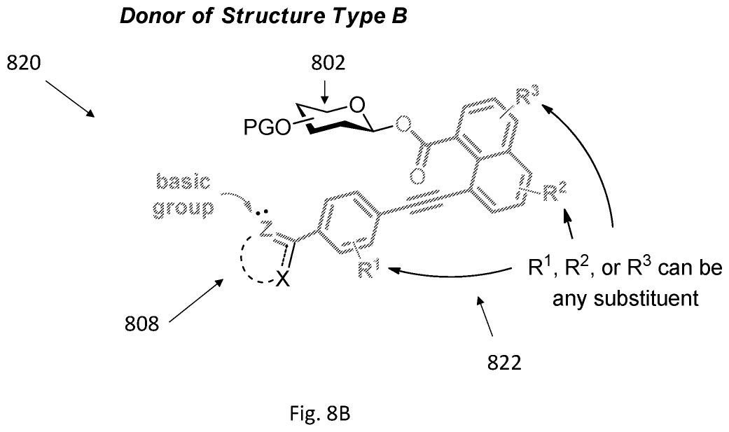

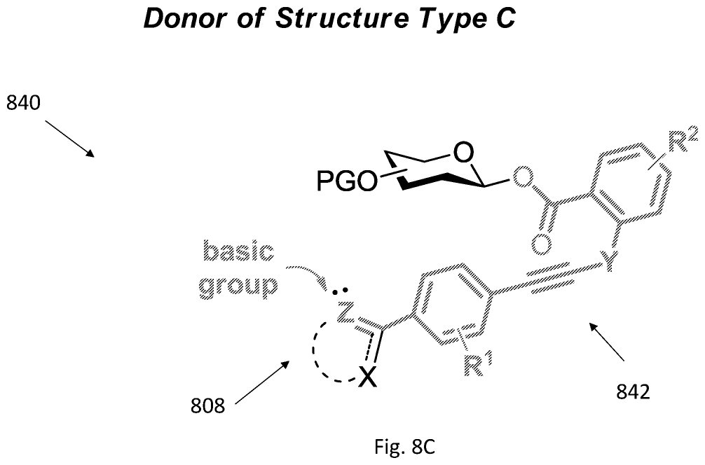

A composition of matter useful as a donor compound in a glycosylation reaction can be embodied in many ways including, but not limited to, the following.
1. FIGS. 8A-8C illustrate example donor compounds 800/820/840 (e.g., glycosyl donor compounds) comprising:
a saccharide moiety 802 covalently bonded to a leaving group 804/822/842, the leaving group comprising a functionality that can be activated by an electrophile 810 (i.e., E⁺) to form an activated leaving group 812 exhibiting electrophilicity; and
a basic group 808 covalently bonded to the leaving group, the basic group forming a hydrogen bond 814 with an acceptor hydroxyl group 815 so that the acceptor hydroxyl group performs a nucleophilic attack breaking the covalent bond 816 between the activated leaving group 812 and the saccharide moiety in a presence of the electrophilicity.
2. FIG. 8A illustrates an example of the composition of matter of example 1, wherein the functionality in the leaving group 804 comprises an ester containing at least one C—C triple bond.
3. FIG. 8B illustrates an example of the composition of matter of example 1 comprising a donor compound 820 wherein the functionality in the leaving group 822 comprises a naphthoate containing at least one C—C triple bond.
4. FIG. 8C illustrates an example of the composition of matter of example 1 comprising the donor compound 840, wherein the functionality in the leaving group 842 comprises an ortho-substituted benzoate containing at least one C—C triple bond.
5. FIGS. 8A-8C illustrate examples of the composition of matter of any of the examples 1-4, wherein the basic group 808 includes a hydrocarbon derivative (e.g., an amide, an oxazole, or a pyridine) comprising at least one of oxygen or nitrogen or any other heteroatom.
6. FIGS. 8A-8C illustrate examples of the composition of matter of any of the examples 1-5 wherein the leaving group 804/822/842 comprises an ester bonded to an alkyne and further to a basic group. The basic group comprises a functional group including a basic atom that can be an oxygen, nitrogen or any other heteroatom.
7. FIGS. 8A-8C illustrate examples of the composition of matter of any of the examples 1-6, wherein the basic group is connected to the alkyne directly or indirectly.
8. The composition of matter of any of the examples 1-7, wherein:
the compound has the structure:

### Donor of Structure Type A

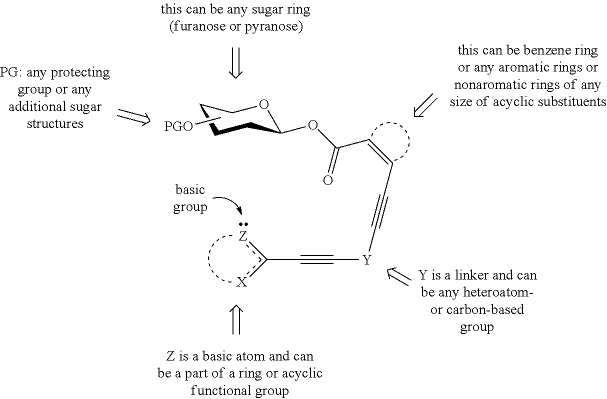

### Donor of Structure Type B

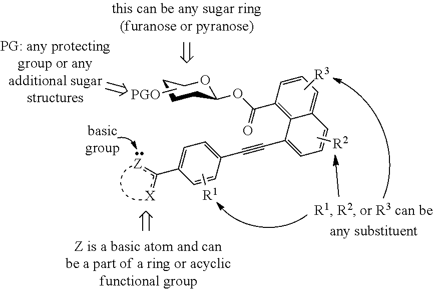

### Donor of Structure Type C

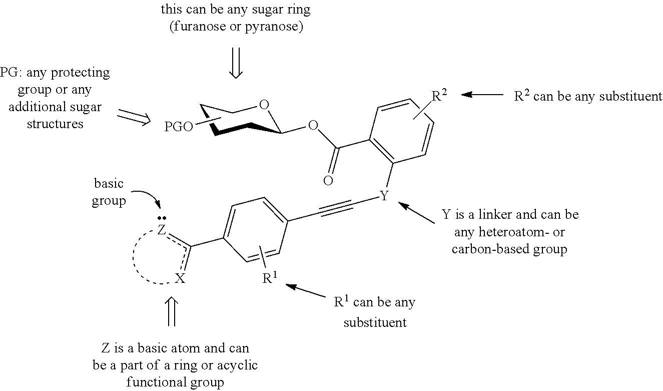 [0154] the saccharide comprises the sugar ring,
Z is a heteroatom, and
X is a heteroatom- or carbon-based group.
Y is a linker and can be any heteroatom- or carbon-based group.
R¹, R² or R³ can be any substituents.
9. FIGS. 8A-8C illustrate the composition of matter of any of the examples 1-8, wherein the saccharide moiety 802 comprises a sugar ring that is 5-membered, 6-membered, or of any ring size (e.g., but not limited to, furanose or pyranose).
10. FIGS. 8A-8C illustrate the composition of matter of any of the examples 1-9, wherein the saccharide moiety 802 comprises a sugar ring comprising any monosaccharide, any oligosaccharide, or any of their modified counterparts.
11. FIGS. 8A-8C illustrate the composition of matter of any of the examples 1-10 wherein the glycosyl donor compound 800/820/840 comprises any of the structures in FIGS. 3-6 or FIG. 3, 4 or 6.
12. FIGS. 8A-8C illustrate the composition of matter of any of the examples 1-11, wherein the glycosyl donor compound 800/820/840 comprises a PG group (PGO).
13. The composition of matter of any of the examples 1-12, wherein the PG group and/or the glycosyl donor compound comprise a disaccharide or oligosaccharide or any sugar moiety.
14. The composition of matter of any of the examples 1-13, wherein the PG group comprises any protecting group or any additional sugar structure.
15. The composition of matter of any of the examples 1-14, wherein the second moiety in the leaving group 804 comprises a component 850 comprising a benzene ring or any aromatic ring or non-aromatic ring of any size or acyclic substituent.
16. The composition of matter of any of the examples 1-15, wherein the leaving group 822 or 842 comprises substituents R¹, R², and/or R³ of any combination of any carbon- or heteroatom-based groups.
17. The composition of matter of any of the examples 1-16, where the leaving group 822 or 842 comprises a linker group Y comprising any carbon- or heteroatom-based group of any length.
18. The composition of matter of any of the examples 1-17, wherein the basic group comprises a phenyl, or steric bulky group, e.g., but not limited to, R1 in FIG. 2C.
19. The composition of matter of any of the examples 1-18, wherein the leaving group comprises a benzoate or aromatic carboxylate group (e.g., but not limited to, as shown in FIG. 2C or 850 in FIG. 8A).
20. The composition of matter of any of the examples 1-19, wherein the composition of the basic group and the leaving group increases selectivity for an alpha or beta type of the carbohydrate.
21. The composition of matter of any of the examples 1-20, wherein the moiety or functionality in the donor leaving group comprises an ester covalently bonded to an alkyne.
22. The composition of any of the examples 1-21, wherein the acceptor compound is any HO group-containing compound.

### Fifth Example: Process Steps for Carbohydrate Synthesis

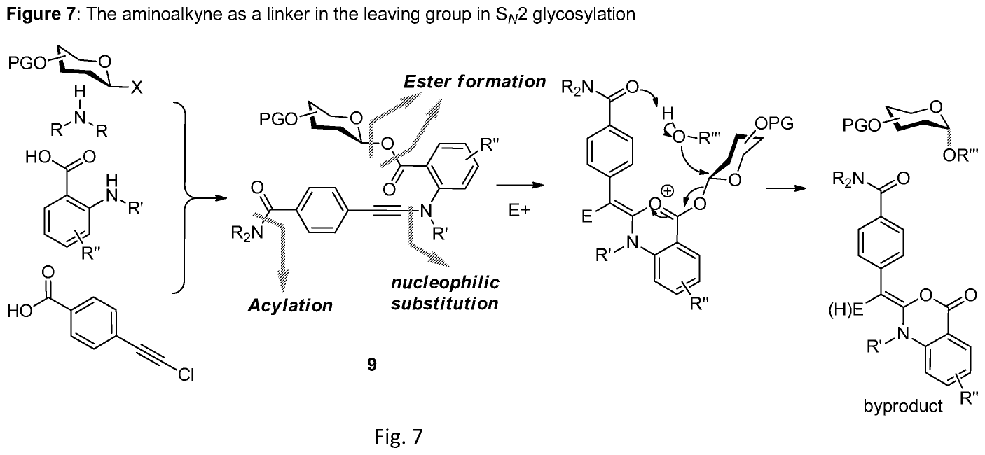

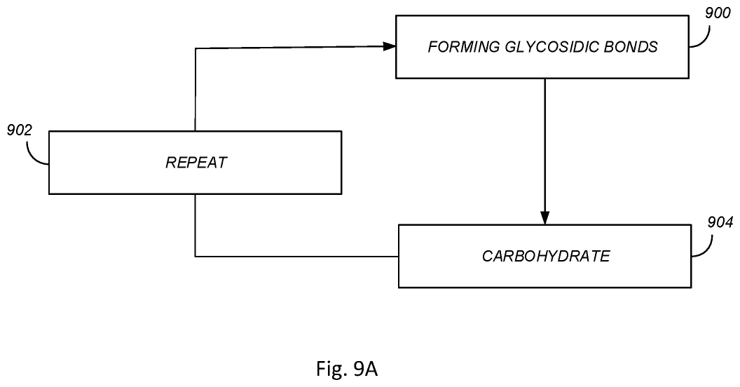

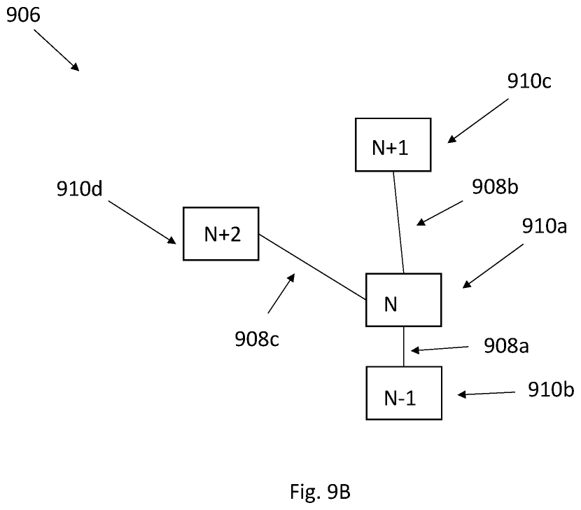

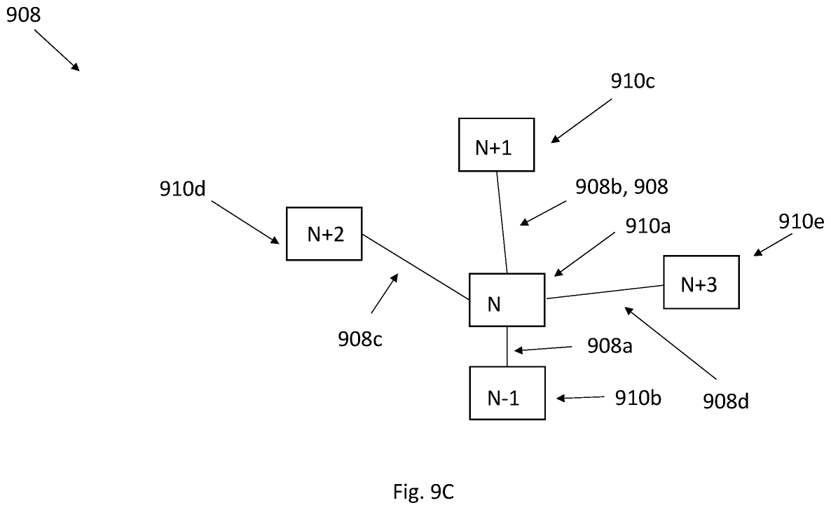

FIG. 9A is a flowchart illustrating example implementations of the SN2 glycosylation strategy for carbohydrate synthesis (referring also to FIGS. 1-9).

Block 900 represents forming one or more glycosidic bonds between a donor compound (e.g., 800, 820, 840, D6 or 9, a glycosyl donor compound donating its saccharide moiety to the oxygen atom of a hydroxyl group in an acceptor compound); and an acceptor compound. The acceptor compound such as 502 comprises the acceptor hydroxyl group 815 and uses its hydroxyl oxygen to accept the saccharide moiety from the glycosyl donor compound. In one or more examples, the acceptor compound is any HO group-containing compound.

The forming steps comprise (i) obtaining a glycosyl donor compound with a structure defined by FIG. 8A-C and comprising the saccharide moiety covalently bonded. to a leaving group; and a basic group covalently bonded to the leaving group; and (ii) activating the leaving group by an electrophile in a presence of an acceptor compound so as to form an activated leaving group in the glycosyl donor compound, and so that the glycosyl donor compound undergoes an SN2 reaction comprising: using FIG. 8A as an example, the basic group 808 on the activated leaving group 812 forming a hydrogen bond with the acceptor hydroxyl group. The formation of the hydrogen bond facilitates a nucleophilic attack by the acceptor hydroxyl group, the nucleophilic attack breaking the covalent bond 816 between the activated leaving group 812 and the saccharide moiety 802; and so as to allow the acceptor hydroxyl group to form a new glycosidic bond with the saccharide moiety 802 in a substitution of the leaving group 804.

In one or more examples, the glycosyl donor compound (e.g., as illustrated in D5 in FIG. 5A) comprises a protecting group covering a hydroxyl group, and the method further comprises exposing the protecting group of the glycoside product to a deprotecting agent that removes the protecting group so as to expose the hydroxyl group prior to forming an additional glycosidic bond with the same or a different glycosyl donor.

In one or more examples, forming the glycosidic bond further comprises providing a catalyst 200 or activation reagent (e.g., an electrophilic halogen) activating the leaving group toward the nucleophilic attack by the acceptor hydroxyl group.

In one or more examples, the forming of the glycosidic bonds comprises combining the donor group and the acceptor group in a solvent comprising an aprotic solvent. In one or more examples, the solvent further comprises a minor less polar component In one or more examples, the forming of the glycosidic bonds comprises combining the donor compound comprising the type A donor or the type B donor and the acceptor compound in a solvent comprising trifluorotoluene.

In one or more examples, the forming of the glycosidic bonds comprises providing a catalyst in a presence of an acid, the catalyst activating the leaving group toward the nucleophilic attack by the acceptor hydroxyl group; the catalyst comprises a metal and the acid reactivates the metal so as to increase a yield of the carbohydrate. In one or more examples, the acid comprises bis(trifluoromethanesulfonyl)imide.

In one or more examples, a composition of the solvent and/or the acid increases a reaction rate of the formation of the glycosidic bonds.

Block 902 represents optionally repeating the forming step of Block 900, e.g., so as to form a carbohydrate comprising an oligosaccharide 504 comprising a chain of a plurality of n saccharides 506 connected by the glycosidic bonds 500, wherein n is an integer. The repeating is such that the carbohydrate formed in the previous step 900 comprises the acceptor compound including the hydroxyl group used to form the glycosidic bond in the next forming step 900. In one or more examples, the oligosaccharide comprises a 1,2-cis glycoside.

In one or more examples, the forming step utilizes the glycosyl donor compound 800/820/840 of any of the examples 1-21 of the fourth example or any of the examples described herein.

Block 904 represents the end result of each glycosylation step, a carbohydrate comprising glycosidic bonds between the saccharide moiety and the hydroxyl group of an acceptor compound.

FIG. 5A illustrates an example wherein the glycosyl donor compound D5 is delivered to the acceptor compound in each of the glycosidic bond forming steps such that, for each of the n saccharides, the glycosidic bonds include a first glycosidic bond connecting the n^{th} saccharide to the (n−1)^{th} saccharide; and a second glycosidic bond connecting the n^{th} saccharide to the (n+1)^{th} saccharide.

FIG. 9B illustrates an example of the synthesis forming the carbohydrate 906 comprising a first glycosidic bond 908a connecting the n^{th} saccharide 910a to the (n−1)^{th} saccharide 910b; a second glycosidic bond 908b connecting the n^{th} saccharide 910a to the (n+1)^{th} saccharide 910c; and a third glycosidic bond 908c connecting the n^{th} saccharide 910a to the (n+2)^{th} saccharide 910d.

FIG. 9C illustrates an example of the synthesis forming the carbohydrate 906 further including a fourth glycosidic bond 908d connecting the n^{th} saccharide 910a to the (n+3)^{th} saccharide 910e.

FIG. 5A and FIG. 5C illustrate examples of the oligosaccharide comprising a branched chain (FIG. 5C) or linear chain (FIG. 5A).

In one or more examples, the forming of the glycosyl bonds comprises a solid phase synthesis (e.g., as described in the Sixth Example). In one or more solid synthesis examples, at least one of the acceptor compounds is connected to a solid support and one or more reagents are delivered to the solid support. In one or more examples, the acceptor compound covalently attached to the solid support (e.g., via a linker structure) is a first acceptor compound in the chain.

In one or more examples, the carbohydrate does not comprise a protecting group covering the hydroxyl group, the protecting group removed by exposure to a deprotecting agent removing the protecting group.

### Sixth Example: Solid Synthesis

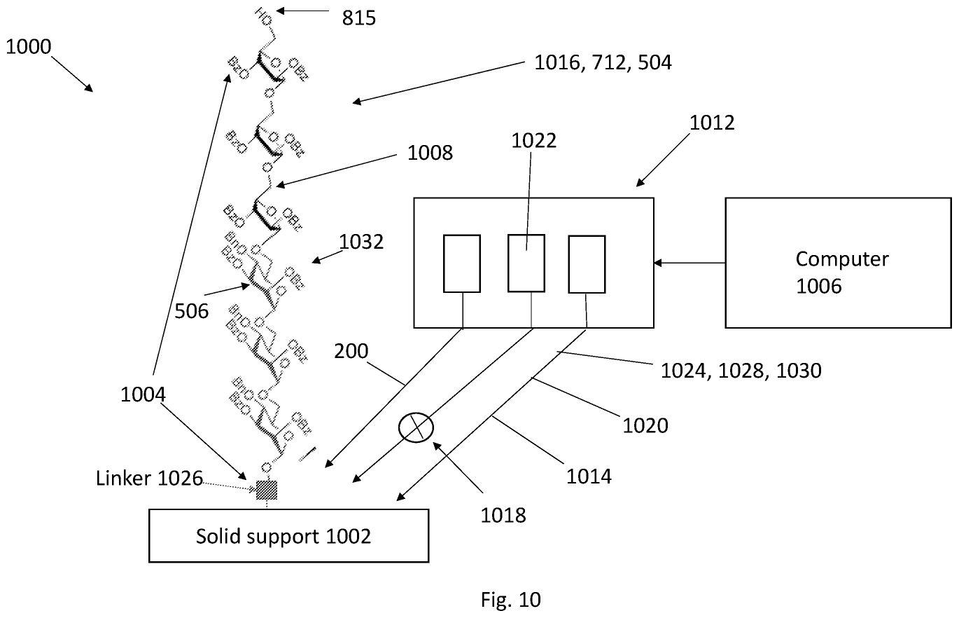

FIG. 10 illustrates a synthesizer for making a carbohydrate using a synthesizer wherein the glycosidic bond forming comprises solid phase synthesis (referring also to FIGS. 1-10). The synthesizer can implement the reactions and processes illustrated in any of the examples described herein (e.g., the fifth example) or illustrated in FIGS. 1-9 or illustrated in FIGS. 1-9, or e.g., using the donor compound of any of the examples 1-22 in the fourth example.

The synthesizer 1000 includes a solid support 1002 for an acceptor compound, 1004 comprising a hydroxyl group 815 (“acceptor hydroxyl group” or HO containing molecule); a computer 1006 executing an algorithm controlling a reaction forming one or more glycosidic bonds 1008, 500, 710 between a donor compound 800/820/840 (e.g., glycosyl donor compound) and the acceptor compound 1004 (e.g., but not limited to glycosyl acceptor compound); and a delivery system 1012 connected to the computer 1006 for delivering reagents 1014 to the solid support in accordance with the algorithm. In this way, the solid support acts as the support for the carbohydrate 1016 as the carbohydrate grows in accordance the reaction controlled by the computer 1006.

In one or more examples, delivery system 1012 includes a system of valves 1018 and tubes 1020 or conduits delivering the reagents from reagent reservoirs 1022 or sources to the solid support.

The reagents delivered by the delivery system include a glycosyl donor compound 800/820/840 comprising the saccharide moiety covalently bonded to a leaving group and a basic group covalently bonded to the leaving group. The reagents further include an electrophilic catalyst 200 (e.g., metal connected to a ligand) or an activator reagent (e.g., an electrophilic halogen source) activating an electrophilicity of the leaving group in a presence of the hydroxyl group of an acceptor. In one or more examples, the acceptor compound 1004 comprises a protecting group covering the hydroxyl group, and the reagents further include a deprotecting agent 1024 removing the protecting group to expose the hydroxyl group for forming the glycosidic bond 1008. In typical examples, the reagents are delivered in liquid or solution phase.

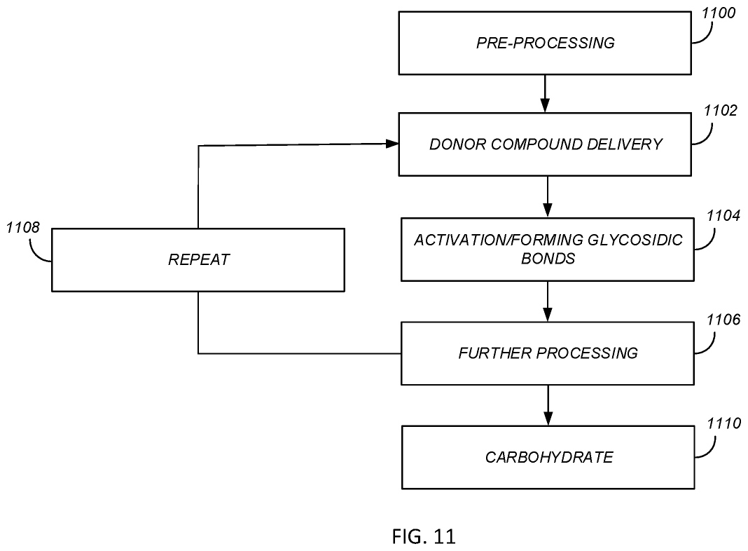

The computer algorithm controls delivery of the reagents in accordance with design of the carbohydrate 1016 being synthesized. In a typical example, the algorithm controls the reaction according to the following steps, as illustrated in FIG. 11.

Block 1100 represents pre-reaction steps including connecting the acceptor compound 1004 to the solid support 1002. This acceptor compound 1004 can be a linker compound 1026 at the beginning. In some examples, the pre-reaction steps include swelling the solid support 1002 with a solvent 1028 or other fluid so that the solid support expands and exposes the acceptor compound 1004 to the liquid phase environment of the reagents delivered in subsequent steps. In various examples, the solid support comprises a polymer (e.g., polystyrene/ polystyrene beads) resin (11), (12).

Block 1102 represents delivering the reagent 1014 including the glycosyl donor compound 800/820/840 to the acceptor compound 1004 connected to the solid support.

Block 1104 represents initiating formation of the glycosidic bond comprising delivering the catalyst 200 or activator reagent to the glycosyl donor compound 800/820/840, so as to activate the nucleophilic part of the leaving group in the glycosyl donor compound 800/820/840 to form an activated leaving group in a presence of the acceptor hydroxyl group. The displacement of this leaving group via an SN2 reaction comprises the basic group on the leaving group directing the nucleophilic attack of the hydroxyl group of the acceptor compound 1004 via a hydrogen bond to form the glycosidic bond 1008. In one or more examples, the activated leaving group electrophilicity allows the reaction comprising an SN2 reaction comprising (1) the basic group on the donor leaving group forming a hydrogen bond with the acceptor hydroxyl group; and (2) the acceptor hydroxyl group performing a nucleophilic attack breaking the covalent bond between the activated leaving group and the saccharide moiety of the glycosyl donor compound 800/820/840; and (3) the acceptor hydroxyl group forming one of the glycosidic bonds 1008 with the saccharide moiety of the glycosyl donor compound 800/820/840 in a SN2 substitution of the activated donor leaving group.

In this way, the carbohydrate comprising one of the glycosidic bonds between the donor saccharide moiety and the acceptor hydroxyl group is made.

Block 1106 represents further processing including washing using a solvent 1030, wherein excess or undesired reagents 1014 are washed away. In one or more examples, the acceptor compound 1004 comprises a protector group covering the hydroxyl group, and the further processing includes delivering a deprotecting agent 1024 to remove the protector group, thereby exposing the hydroxyl group being used to form the next glycosidic bond.

Block 1108 represents repeating steps 1102-1106. In a typical example, carbohydrate being synthesized is an oligosaccharide including a chain of the saccharides connected by the glycosidic bonds and the computer algorithm controls the delivery of reagents so as to repeat the glycosidic bond forming step 1104 such that:
the carbohydrate formed in a previous step 1104 comprises the acceptor compound including the hydroxyl group for the next forming step 1104;
a glycosyl donor compound 800/820/840 is delivered to the acceptor compound in each of the forming steps, so that for each of a plurality of n saccharides 1032 in the chain (n is an integer), the glycosidic bonds include a first glycosidic bond connecting the n^{th} saccharide to the (n−1)^{th} saccharide; and a second glycosidic bond connecting the n^{th} saccharide to the (n+1)^{th} saccharide (e.g. as illustrated in FIG. 10 or 5A). In some examples, the glycosidic bonds further include a third glycosidic bond connecting the n^{th} saccharide to the (n+2)^{th} saccharide (e.g., as illustrated in FIG. 9B). In yet further examples, the glycosidic bonds further include a fourth glycosidic bond connecting the n^{th} saccharide to the (n+3)^{th} saccharide (e.g., as illustrated in FIG. 9C).

In various examples, the acceptor compound is originally a donor or coming from a donor including protected HO groups (hydroxyl group). Once the donor is ‘donated’ or reacted in the glycosylation chemistry, it is no longer a donor (as the leaving group is gone) and becomes a part of the carbohydrate molecule. If the donor's protected HO group is deprotected, in other words, becoming the free HO group, it can behave as an acceptor accepting a donor's sugar moiety to make the new glycosidic bond. Thus, in the context of making oligosaccharides, the donor later becomes a part of the acceptor.

In various examples, the glycosidic bonds formed in the process may comprise 1,2-cis glycosidic linkage in the formed oligosaccharide, e.g., so that the oligosaccharide comprises a 1,2-cis glycoside.

In various examples, the oligosaccharide formed using the method comprises a linear or branched connections of monosaccharide molecules.

Block 1110 represents the end result, an oligosaccharide.

In various examples, the solid synthesis or synthesizer 1000 operates using the glycosyl donor compound according to examples described herein and the methods and apparatus described in “Automated glycan assembly using the Glyconeer 2.1 synthesizer,” www.pnas.org/cgi/doi/10.1073/pnas.170014914, which publication is incorporated by reference herein.

In one or more examples, the acceptor compound comprises a protecting group such as the acetyl 501 covering the hydroxyl group and the delivery system further delivers a deprotecting agent such as NaOMe 503 removing the protecting group so as to expose the hydroxyl group and form the carbohydrate without the protecting group.

In one or more examples, the acceptor compound is any HO group-containing compound.

### Example Hardware Environment

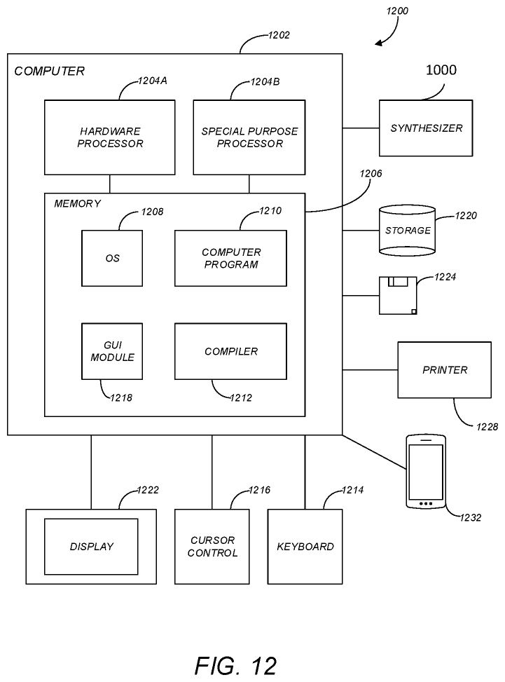

FIG. 12 is an exemplary hardware and software environment 1200 (referred to as a computer-implemented system and/or computer-implemented method) used to implement one or more embodiments of the invention. The hardware and software environment includes a computer 1202 and may include peripherals. Computer 1202 may be a user/client computer, server computer, or may be a database computer. The computer 1202 comprises a hardware processor 1204A and/or a special purpose hardware processor 1204B (hereinafter alternatively collectively referred to as processor 1204) and a memory 1206, such as random access memory (RAM). The computer 1202 may be coupled to, and/or integrated with, other devices, including input/output (I/O) devices such as a keyboard 1214, a cursor control device 1216 (e.g., a mouse, a pointing device, pen and tablet, touch screen, multi-touch device, etc.) and a printer 1228. In one or more embodiments, computer 1202 may be coupled to, or may comprise, a portable device 1232 (e.g., cellular device, personal digital assistant, etc. laptop, tablet, multi-touch device, or internet enabled device executing on various platforms and operating systems).

In one embodiment, the computer 1202 operates by the hardware processor 1204A performing instructions defined by the computer program 1210 (e.g., algorithms for controlling reactions and synthesizer as described herein) under control of an operating system 1208. The computer program 1210 and/or the operating system 1208 may be stored in the memory 1206 and may interface with the user and/or other devices to accept input and commands and, based on such input and commands and the instructions defined by the computer program 1210 and operating system 1208, to provide output and results.

Output/results may be presented on the display 1222 or provided to another device for presentation or further processing or action. The image may be provided through a graphical user interface (GUI) module 1218. Although the GUI module 1218 is depicted as a separate module, the instructions performing the GUI functions can be resident or distributed in the operating system 1208, the computer program 1210, or implemented with special purpose memory and processors.

Some or all of the operations performed by the computer 1202 according to the computer program 1210 instructions may be implemented in a special purpose processor 1204B. In this embodiment, some or all of the computer program 1210 instructions may be implemented via firmware instructions stored in a read only memory (ROM), a programmable read only memory (PROM) or flash memory within the special purpose processor 1204B or in memory 1206. The special purpose processor 1204B may also be hardwired through circuit design to perform some or all of the operations to implement the present invention. Further, the special purpose processor 1204B may be a hybrid processor, which includes dedicated circuitry for performing a subset of functions, and other circuits for performing more general functions such as responding to computer program 1210 instructions. In one embodiment, the special purpose processor 1204B is an application specific integrated circuit (ASIC) or field programmable gate array (FPGA).

The computer 1202 may also implement a compiler 1212 that allows an application or computer program 1210 written in a programming language such as C, C++, Assembly, SQL, PYTHON, PROLOG, MATLAB, RUBY, RAILS, HASKELL, or other language to be translated into processor 1204 readable code. Alternatively, the compiler 1212 may be an interpreter that executes instructions/source code directly, translates source code into an intermediate representation that is executed, or that executes stored precompiled code. Such source code may be written in a variety of programming languages such as JAVA, JAVASCRIPT, PERL, BASIC, etc. After completion, the application or computer program 1210 accesses and manipulates data accepted from I/O devices and stored in the memory 1206 of the computer 1202 using the relationships and logic that were generated using the compiler 1212.

The computer 1202 also optionally comprises an external communication device such as a modem, satellite link, Ethernet card, or other device for accepting input from, and providing output to, other computers 1202.

In one embodiment, instructions implementing the operating system 1208, the computer program 1210, and the compiler 1212 are tangibly embodied in a non-transitory computer-readable medium, e.g., data storage device 1220, which could include one or more fixed or removable data storage devices, such as a zip drive, floppy disc drive 1224, hard drive, CD-ROM drive, tape drive, etc. Further, the operating system 1208 and the computer program 1210 are comprised of computer program 1210 instructions which, when accessed, read and executed by the computer 1202, cause the computer 1202 to perform the steps necessary to implement and/or use the present invention or to load the program of instructions into a memory 1206. thus creating a special purpose data structure causing the computer 1202 to operate as a specially programmed computer executing the method steps described herein. Computer program 1210 and/or operating instructions may also be tangibly embodied in memory 1206 and/or data communications devices 1230, thereby making a computer program product or article of manufacture according to the invention. As such, the terms “article of manufacture,” “program storage device,” and “computer program product,” as used herein, are intended to encompass a computer program accessible from any computer readable device or media.

Of course, those skilled in the art will recognize that any combination of the above components, or any number of different components, peripherals, and other devices, may be used with the computer 1202.

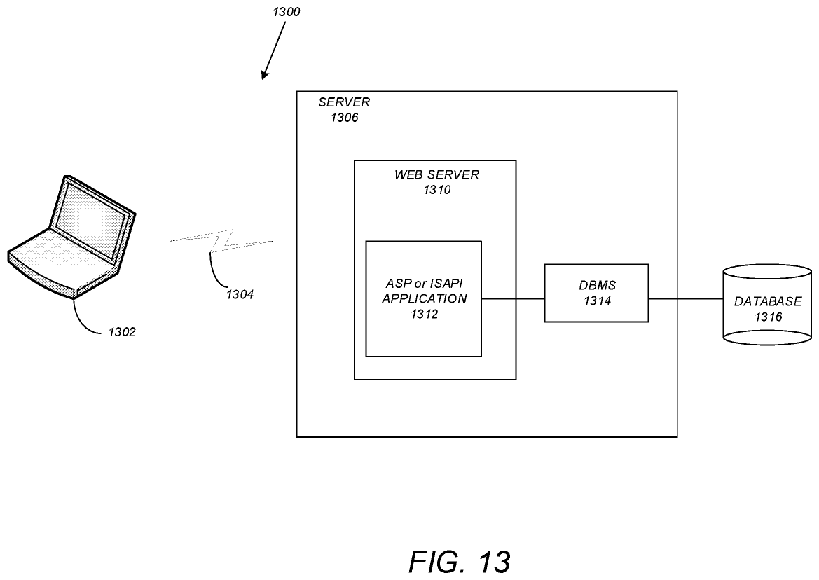

FIG. 13 schematically illustrates a typical distributed/cloud-based computer system 1300 using a network 1304 to connect client computers 1302 to server computers 1306. A typical combination of resources may include a network 1304 comprising the Internet, LANs (local area networks), WANs (wide area networks), SNA (systems network architecture) networks, or the like, clients 1302 that are personal computers or workstations (as set forth in FIG. 12), and servers 1306 that are personal computers, workstations, minicomputers, or mainframes (as set forth in FIG. 12). However, it may be noted that different networks such as a cellular network (e.g., GSM [global system for mobile communications] or otherwise), a satellite based network, or any other type of network may be used to connect clients 1302 and servers 1306 in accordance with embodiments of the invention.

A network 1304 such as the Internet connects clients 1302 to server computers 1306. Network 1304 may utilize ethernet, coaxial cable, wireless communications, radio frequency (RF), etc. to connect and provide the communication between clients 1302 and servers 1306. Further, in a cloud-based computing system, resources (e.g., storage, processors, applications, memory, infrastructure, etc.) in clients 1302 and server computers 1306 may be shared by clients 1302, server computers 1306, and users across one or more networks. Resources may be shared by multiple users and can be dynamically reallocated per demand. In this regard, cloud computing may be referred to as a model for enabling access to a shared pool of configurable computing resources.

Clients 1302 may execute a client application or web browser and communicate with server computers 1306 executing web servers 1310. Such a web browser is typically a program such as MICROSOFT INTERNET EXPLORER/EDGE, MOZILLA FIREFOX, OPERA, APPLE SAFARI, GOOGLE CHROME, etc. Further, the software executing on clients 1302 may be downloaded from server computer 1306 to client computers 1302 and installed as a plug-in or ACTIVEX control of a web browser. Accordingly, clients 1302 may utilize ACTIVEX components/component object model (COM) or distributed COM (DOOM) components to provide a user interface on a display of client 1302. The web server 1310 is typically a program such as MICROSOFT'S INTERNET INFORMATION SERVER.

Web server 1310 may host an Active Server Page (ASP) or Internet Server Application Programming Interface (ISAPI) application 1312, which may be executing scripts. The scripts invoke objects that execute business logic (referred to as business objects). The business objects then manipulate data in database 1316 through a database management system (DBMS) 1314. Alternatively, database 1316 may be part of, or connected directly to, client 1302 instead of communicating/obtaining the information from database 1316 across network 1304. When a developer encapsulates the business functionality into objects, the system may be referred to as a component object model (COM) system. Accordingly, the scripts executing on web server 1310 (and/or application 1312) invoke COM objects that implement the business logic. Further, server 1306 may utilize MICROSOFT'S TRANSACTION SERVER (MTS) to access required data stored in database 1316 via an interface such as ADO (Active Data Objects), OLE DB (Object Linking and Embedding DataBase), or ODBC (Open DataBase Connectivity).

Generally, these components 1300-1316 all comprise logic and/or data that is embodied in/or retrievable from device, medium, signal, or carrier, e.g., a data storage device, a data communications device, a remote computer or device coupled to the computer via a network or via another data communications device, etc. Moreover, this logic and/or data, when read, executed, and/or interpreted, results in the steps necessary to implement and/or use the present invention being performed.

Although the terms “user computer”, “client computer”, and/or “server computer” are referred to herein, it is understood that such computers 1302 and 1306 may be interchangeable and may further include thin client devices with limited or full processing capabilities, portable devices such as cell phones, notebook computers, pocket computers, multi-touch devices, and/or any other devices with suitable processing, communication, and input/output capability.

Of course, those skilled in the art will recognize that any combination of the above components, or any number of different components, peripherals, and other devices, may be used with computers 1302 and 1306. Embodiments of the invention are implemented as a software application on a client 1302 or server computer 1306. Further, as described above, the client 1302 or server computer 1306 may comprise a thin client device or a portable device that has a multi-touch-based display.

Thus, FIGS. 12-13 illustrate a computer implemented system, comprising one or more processors; one or more non-transitory memories; and one or more programs stored in the one or more non-transitory memories, wherein the one or more programs executed by the one or more processors controlling a reaction forming one or more glycosidic bonds between a donor and the acceptor, the reaction comprising an SN2 reaction comprising the basic group on the leaving group forming a hydrogen bond with the acceptor hydroxyl group; the acceptor hydroxyl group performing a nucleophilic attack breaking a covalent bond between the activated leaving group and the saccharide moiety; and the acceptor hydroxyl group forming one of the glycosidic bonds with the saccharide moiety in a substitution of the activated leaving group; so that the carbohydrate comprising the one of the glycosidic bonds between the saccharide moiety and the acceptor hydroxyl group is made.

### Advantages and Improvements

Embodiments of the present invention include the following advantages:
a) Providing a generally applicable glycosylation strategy with control of anomeric stereochemistry—no such a chemistry is currently available in academia or in commercial space for the synthesis of 1,2-cis glycosides, 2-deoxy glycosides, sialosides, and other sugar types.
b) Empowering the development of automatic carbohydrate synthesis. Such an automatic synthesis can be offered as a service-on-demand—as in the business of peptide or nucleic acid synthesis—and would greatly facilitate research in glycoscience, which is relevant in combating many diseases and infections (including COVID-19). Alternatively, this enabling technology can be developed into marketable glyco synthesizers, e.g., such as glycouniverse (https://glycouniverse.com/). The chemistry would greatly improve the commercial potential of the market by making more complex and demanding oligosaccharides available.
c) The commercial success realized by the automatic peptide and nucleic acid synthesis technologies evidences the utility of the methods described herein.

### REFERENCES

The following references are incorporated by reference herein.
(1) Vilotijevic, I.; Götze, S.; Seeberger, P.; Silva, D. Modern Synthetic Methods in Carbohydrate Chemistry; Wiley-VCH, 2013.
(2) Werz, D. B.; Vidal, S. Modern Synthetic Methods in Carbohydrate Chemistry: From Monosaccharides to Complex Glycoconjugates, p 1 online resource (xxiii, 381 pages).
(3) Glycoscience Chemistry and Chemical Biology; Fraser-Reid, B. O.; Tatsuta, K.; Thiem, J.; SpringerLink (Online service), Eds.; Springer-Verlag Berlin Heidelberg: Berlin, Heidelberg, 2008.
(4) Adero, P. O.; Amarasekara, H.; Wen, P.; Bohé, L.; Crich, D. ‘The Experimental Evidence in Support of Glycosylation Mechanisms at the Sn1-Sn2 Interface’ Chem. Rev. 2018, 98, 8242-8284.
(5) Demchenko, A. V. ‘1,2-Cis O-Glycosylation: Methods, Strategies, Principles’ Curr. Org. Chem. 2003, 7, 35-79.
(6) Nigudkar, S. S.; Denichenko, A. V. ‘Stereocontrolled 1,2-Cis Glycosylation as the Driving Force of Progress in Synthetic Carbohydrate Chemistry’ Chem. Sci. 2015, 6, 2687-2704.
(7) Yu, B. ‘Gold(I)-Catalyzed Glycosylation with Glycosyl O-Alkynylbenzoates as Donors’ Acc. Chem. Res. 2018, 51, 507-516.
(8) Zhu, Y.; Yu, B, ‘Characterization of the Isochromen-4-Y1-Gold(I) Intermediate in the Gold(I)-Catalyzed Glycosidation of Glycosyl Ortho-Alkynylbenzoates and Enhancement of the Catalytic Efficiency Thereof’ Angew. Chem., Int. Ed. 209, n/a-n/a.
(9) Kawade, R. K.; Liu, R.-S. ‘Gold-Catalyzed Oxidative Cyclization of 4-Allenyl-1-Ynes with 8-Methylquinoline Oxide’ 2013, 15, 4094-4097.
(10) Crich, D. ‘Chemistry of Glycosyl Triflates: Synthesis of B-Mannopyranosides’ J. Carbohydr. Chem. 2002, 21, 663-686.
(11) Seeberger, P. H. ‘Automated Oligosaccharide Synthesis’ Chemical Society Review 2008, 37, 19-28.

### Danglad-Flores, J.; Leichnitz, S.;

(12) Sletten, E. T.; Abragam Joseph, A.; Bienert, K.; Le Mai Hoang, K.; Seeberger, P. H. ‘Microwave-Assisted Automated Glycan Assembly’ Journal of the American Chemical Society 2021, 143, 8893-8901.

### CONCLUSION

This concludes the description of the preferred embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A method for making a carbohydrate, comprising:
(a) forming one or more glycosidic bonds between a glycosyl donor compound and an acceptor compound comprising one or more hydroxyl group, comprising:
(i) obtaining the glycosyl donor compound comprising:
a saccharide moiety covalently bonded to a leaving group; and
a basic group covalently bonded to the leaving group;
(ii) activating the leaving group by an electrophile in a presence of the acceptor compound so as to form an activated leaving group in the glycosyl donor compound undergoing an SN2 reaction comprising:
the basic group forming a hydrogen bond with the acceptor hydroxyl group; and
formation of the hydrogen bond facilitating a nucleophilic attack by the acceptor hydroxyl group, the nucleophilic attack breaking the covalent bond between the donor activated leaving group and the donor saccharide moiety; and
the acceptor hydroxyl group forming the one of the glycosidic bonds with the donor saccharide moiety in a substitution of the leaving group;
so that the carbohydrate comprising the one of the glycosidic bonds between the donor saccharide moiety and the acceptor hydroxyl group is made.

2. The method of claim 1, wherein the carbohydrate comprises an oligosaccharide comprising a chain of a plurality of n saccharides connected by the glycosidic bonds, wherein n is an integer, the method further comprising:
repeating the forming step (a) such that:
the carbohydrate formed in the previous step (a) comprises the acceptor compound including the hydroxyl group used to form the one of the glycosidic bonds in the next forming step (a);
the glycosyl donor compound is delivered to the acceptor compound in each of the forming steps such that, for each of the n saccharides, the glycosidic bonds include:
a first glycosidic bond connecting the n^{th} saccharide to the (n−1)^{th} saccharide; and
a second glycosidic bond connecting the n^{th} saccharide to the (n+1)^{th} saccharide.

3. The method of claim 2, wherein the glycosidic bonds further include a third glycosidic bond connecting the n^{th} saccharide to the (n+2)^{th} saccharide.

4. The method of claim 3, wherein the glycosidic bonds further include a fourth glycosidic bond connecting the n^{th} saccharide to the (n+3)^{th} saccharide.

5. The method of claim 1, wherein:
the leaving group comprises an ester bonded to an alkyne and
the basic group comprises a functional group including a basic atom comprising a heteroatom.

6. The method of claim 1, wherein:
the glycosyl donor compound comprises a donor structure type A, a donor structure type B, or a donor structure type C and:
Donor of Structure Type A 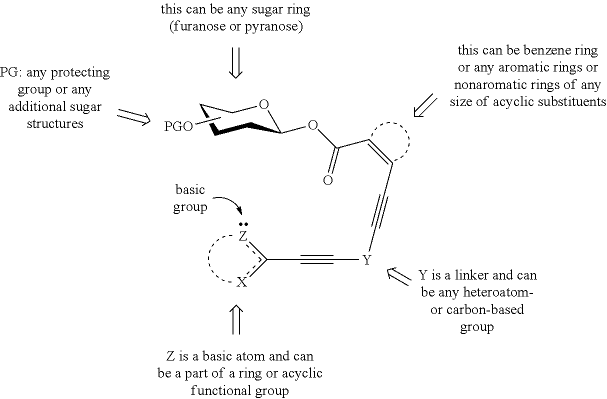
Donor of Structure Type B 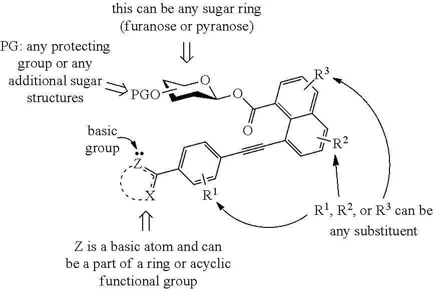
Donor of Structure Type C 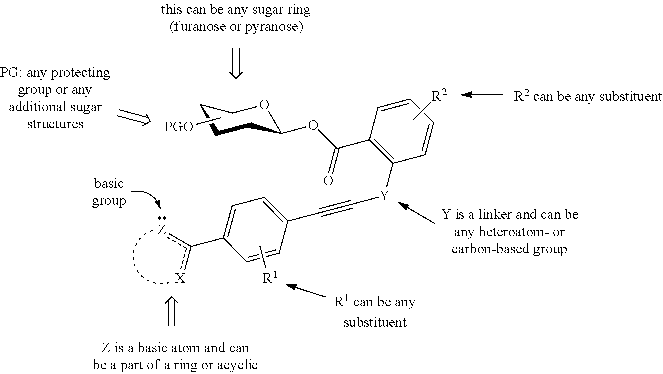
the saccharide moiety comprises a sugar ring;
Z is a heteroatom,
X is a heteroatom or a carbon-based group,
Y is a linker and can be any heteroatom- or carbon-based group, and
R¹, R² or R³ can be any substituents.

7. The method of claim 1, wherein the saccharide moiety comprises any monosaccharide, any oligosaccharide, or any of their modified counterparts.

8. The method of claim 1, wherein the acceptor compound comprises a protecting group covering the hydroxyl group, the method further comprising exposing the protecting group to a deprotecting agent removing the protecting group so as to expose the hydroxyl group prior to forming the one of the glycosidic bonds with the hydroxyl group.

9. The method of claim 2, wherein the oligosaccharide comprises a branched or linear chain.

10. The method of claim 1, wherein the forming of the glycosidic bonds comprises a solid phase synthesis.

11. The method of claim 1, wherein:
at least one of the acceptor compounds is connected to a solid support and one or more reagents are delivered to the solid support.

12. The method of claim 11, wherein the acceptor compound covalently attached to the solid support is a first acceptor compound in the chain.

13. The method of claim 11, wherein the acceptor compound is connected to the solid support via a linker structure.

14. The method of claim 12, wherein the solid support comprises a polymer or resin.

15. The method of claim 1, wherein the forming of the glycosidic bonds further comprises providing a catalyst or activation reagent activating the leaving group toward the nucleophilic attack by the acceptor hydroxyl group.

16. The method of claim 2, wherein the oligosaccharide comprises a 1,2-cis glycoside.

17. A carbohydrate synthesized according to method of claim 1.

18. The carbohydrate of claim 17, wherein the carbohydrate does not comprise a protecting group covering the hydroxyl group, the protecting group removed by exposure to a deprotecting agent removing the protecting group.

19. A synthesizer for making a carbohydrate, comprising:
a solid support for an acceptor comprising a hydroxyl group (“acceptor hydroxyl group”);
a computer executing an algorithm controlling a reaction forming one or more glycosidic bonds between a donor and the acceptor; and
a delivery system for delivering reagents to the solid support in accordance with the algorithm, the reagents including:
a donor comprising:
a saccharide moiety covalently bonded to a leaving group; and
a basic group covalently bonded to the leaving group;
an electrophilic catalyst or activation reagent activating an electrophilicity of the leaving group so as to form an activated leaving group in a presence of the acceptor hydroxyl group, the electrophilicity allowing the reaction comprising an SN2 reaction comprising:
the basic group on the leaving group forming a hydrogen bond with the acceptor hydroxyl group; and
the acceptor hydroxyl group performing a nucleophilic attack breaking a covalent bond between the donor activated leaving group and the donor saccharide moiety;
the acceptor hydroxyl group forming one of the glycosidic bonds with the donor saccharide moiety in a substitution of the activated leaving group;
so that the carbohydrate comprising the one of the glycosidic bonds between the donor saccharide moiety and the acceptor hydroxyl group is made.

20. The synthesizer of claim 19 configured to perform the method of claim 1.

21. The synthesizer of claim 19, wherein the acceptor compound comprises a protecting group covering the hydroxyl group and the delivery system further delivers a deprotecting agent removing the protecting group so as to expose the hydroxyl group and form the carbohydrate without the protecting group.

22.-28. (canceled)

29. The synthesizer of claim 1, wherein the basic group comprises a phenyl, or steric bulky group, the leaving group comprises a benzoate or aromatic carboxylate group, the forming of the glycosidic bonds comprises combining the donor group and the acceptor group in a solvent comprising at least one of an aprotic solvent or a minor less polar component.

30.-38. (canceled)
